(19) United States
(12) Patent Application Publication
(10) Pub. No.: US 2026/0049157 A1
(43) Pub. Date: Feb. 19, 2026

(54) Anti-MuSK Agonistic Antibodies

(71) Applicant: Mabwell Therapeutics, Inc., San Diego, CA (US)

(72) Inventors: Yubin Wang, San Diego, CA (US); Lei Huang, San Diego, CA (US); Xin Du, La Jolla, CA (US)

(21) Appl. No.: 19/265,808

(22) Filed: Jul. 10, 2025

Related U.S. Application Data
(60) Provisional application No. 63/669,834, filed on Jul. 11, 2024.

(51) Int. Cl. C07K 16/40; A61K 39/00

(52) U.S. Cl. CPC C07K 16/40; A61K 2039/505; C07K 2317/24; C07K 2317/52; C07K 2317/565; C07K 2317/71; C07K 2317/75; C07K 2317/92

(57) ABSTRACT

The present disclosure relates to anti-Muscle-Specific Kinase (MuSK) antibodies, in particular agonistic ant-MuSK antibodies, useful to restore or increase the function of the neuromuscular junction (NMJ).

### CROSS-REFERENCE

This application claims priority to U.S. Provisional Patent Application No. 63/669,834, filed on Jul. 11, 2024, the contents of which are incorporated herein by reference in their entirety.

### SEQUENCE LISTING

The computer-readable Sequence Listing submitted on Jul. 10, 2025, and identified as follows: 145,112 bytes ST.26 XML document file named “Sequence Listing.xml,” created Jul. 9, 2025, is incorporated herein by reference in its entirety.

### BACKGROUND

The present disclosure relates generally to antibodies that bind to and activate Muscle-Specific Kinase (MuSK).

MuSK is a receptor tyrosine kinase crucial for the function of neuromuscular junction (NMJ). Hubbard et al., Biochimica et Biophysica Acta (BBA)-Proteins and Proteomics, 1834 (10): 2166-2169 (2013). MuSK is essential during early stages of NMJ development, mediating the clustering of acetylcholine receptors (AChRs) on NMJ, which is essential for the formation and maintenance of functional NMJ. DeChiara et al., Cell, 85:501-512 (1996); Glass et al., Cell, 85:513-523 (1996); Flanagan-Steet et al., Development, 132:4471-4481 (2005); Panzer et al., J. Neurosci., 26:934-947 (2006).

MuSK is a single-pass transmembrane glycoprotein of ˜120 kDa with an extracellular region comprising three Ig-like domains (Ig1, Ig2, Ig3) and one frizzled-like cysteine-rich domain (Fz-CRD). A single transmembrane helix connects the extracellular domains to the intracellular tyrosine kinase domain. Its intracellular region contains several tyrosine phosphorylation sites which are crucial for MuSK activation. Hubbard et al., Biochimica et Biophysica Acta, 1834:2166-2169 (2013).

MuSK is expressed on the membrane of muscle cells. Zhu et al., J. Neurosci., 28 (7): 1688-1696 (2008). Activation of MuSK by agrin, a neuronally derived heparin sulfate proteoglycan, and lipoprotein-related protein 4 (LRP4), the agrin receptor, leads to clustering of acetylcholine receptors (AChRs) on the postsynaptic side of the NMJ, enabling neuromuscular transmission and muscle contraction. Recessive mutations in agrin, LRP4, or MuSK genes cause congenital myasthenia, a group of neuromuscular disorders that compromise the structure and function of neuromuscular synapses and lead to muscle weakness and fatigue.

The literature identifies that MuSK activation is also frustrated by pathogenic autoantibodies and/or defects in the agrin-LPR4-MuSK pathway leading to NMJ dysfunction and neuromuscular disease. Gilhaus et al., Lancet Neurol., 14:1023-1036 (2015); Arimura et al., Science, 345 (6203): 1505-1508 (2014). Thus, a need exists for therapies that overcome these challenges and restore NMJ function. For example, a need exists for antibodies that are capable of binding and activating the signaling and/or phosphorylation of MuSK.

### BRIEF DESCRIPTION OF THE FIGURES

FIG. 1. FIG. 1A is a western blot analysis of MuSK phosphorylation in C2C12 myotubes treated with anti-MuSK agonistic antibodies; and FIG. 1B is a quantification of the ratio of phospho-MuSK to total MuSK (N=3).

FIG. 2. FIG. 2A is images of AChR clustering in C2C12 myotubes treated with a representative anti-MuSK agonistic antibody and controls; FIG. 2B is a quantification of AChR cluster density in C2C12 myotubes treated with a representative anti-MuSK agonistic antibody and controls (N=4); and FIG. 2C is a quantification of percentage area of AChR clusters in C2C12 myotubes treated with a representative anti-MuSK agonistic antibody and controls (N=4).

FIG. 3. Plot of MuSK phosphorylation and AChR clustering in C2C12 stimulated by various anti-MuSK agonistic antibodies.

FIG. 4. Representative images of IHC of NMJ in mouse diaphragm muscle with alpha-bungarotoxin and anti-human IgG. Human anti-MuSK agnostic antibodies were injected intraperitoneally (0.1, 1, and 10 mg/kg) to C57BL/6J mice at postnatal day 4. Diaphragm muscle was collected 3 days following injection.

FIG. 5. Quantification of the ratios of human IgG fluorescence intensity to alpha-bungarotoxin on NMJ. N=3-4 (3-4 mice at each dose).

### DETAILED DESCRIPTION

This description is not provided to limit the disclosure to the embodiments described herein, but rather to explain and teach various principles to enable one of ordinary skill in the art to understand these principles and, with that understanding, be able to apply them to practice not only the embodiments described herein, but also other embodiments that may come to mind in accordance with these principles. The scope of the instant disclosure is intended to cover all such embodiments that may fall within the scope of the appended claims, either literally or under the doctrine of equivalents. All references cited in the present disclosure are incorporated by reference in their entirety.

### Definitions

The following terms or definitions are provided solely to aid in the understanding of the present disclosure. Unless specifically defined herein, all terms used herein have the same meaning as would be understood to one of ordinary skill in the art.

Unless indicated otherwise, all methods, steps, and techniques that are not specifically described in detail can be performed and have been performed in a manner known to a skilled artisan and described in the prior art, such as Sambrook et al., Molecular Cloning: A Laboratory Manual 2^{nd} ed., Cold Spring Harbor Press, Plainsview, NY (1989) and Ausubel et al., Current Protocols in Molecular Biology (Supplement 47), John Wiley & Sons, NY (1999).

As used herein, the singular forms “a”, “an”, and “the” include both singular and plural unless the context clearly indicates otherwise.

“Antibody” refers in the broadest sense to a polypeptide or combination of polypeptides that recognizes and binds to an antigen through one or more immunoglobulin variable regions, where the immunoglobulin variable regions may be naturally occurring or non-naturally occurring, e.g., as a result of engineering, chimerization, humanization, optimization, CDR-grafting, or affinity maturation.

An “antibody” as disclosed herein can be a whole (intact, full length) antibody, a single chain antibody, or an antigen binding fragment with one or two chains. An antibody comprises at least sufficient complementarity determining regions (CDR), interspersed with framework regions (FR), for the antibody to recognize and bind to an antigen. An anti-MuSK antibody disclosed herein may be, but is not limited to, at least one of a monoclonal antibody, a polyclonal antibody, a humanized antibody, a fully human antibody, a chimeric antibody, a single chain antibody, a Fab fragment, a single-chain variable fragment (scFv), an aptamer, a single-domain antibody (VHH or nanobody), a recombinant antibody, a modified antibody having peptide/other moieties attached to antibody and/or additional amino acids added the N- or C-terminus, or other MuSK fragment or variant. Whole antibody, full length antibody, intact antibody, or equivalent terms are understood to refer to a polypeptide, in particular a glycoprotein, comprising at least two heavy chains (HCs) and two light chains (LCs) interconnected by disulfide bonds. Each HC is comprised of a heavy chain variable region (VH) and an HC constant region (CH), and each light chain is comprised of a light chain variable region (VL) and an LC constant region (CL). The HC and LC variable regions, VH and VL, include a binding domain that interacts with an antigen. The VH and VL regions can be further subdivided into CDR regions characterized by hypervariability, interspersed with FR regions that are typically more conserved. Each VH and VL is typically composed of three CDRs and four FRs arranged from amino-terminus to carboxy-terminus in the following order: FR1, CDR1, FR2, CDR2, FR3, CDR3, FR4. The constant regions of the antibodies may mediate the binding of the immunoglobulin to host tissues or factors, including various cells of the immune system and the classical complement system. “Anti-MuSK antibody” and “Anti-MuSK agonistic antibody” are terms used interchangeably herein.

As used herein, the term “sequence identity” is defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein. By way of example, if a first sequence is identical to a second sequence, they have 100% sequence identity; and if a first sequence contains 100 residues and a second sequence has 100 residues, but differs in only on residue, they have 99% sequence identity. Software tools such as NCBI Blast (Basic Local Alignment Search Tool) and others provide the sequence identity as referred to in the present disclosure. Assessment of sequence identity may also be conducted on part of a provided sequence. For example, in embodiments, “part of a provided sequence” may mean at least 50% of the length of the SEQ ID NO, or at least 60%, or at least 70%, or at least 80%, or at least 90% after the respective sequence alignment is performed. Unless specified, “sequence identity” is being referred to in the context of the entire SEQ ID NO.

As used herein, a “conservative amino acid substitution” is an amino acid substitution in which an amino acid residue is replaced with another amino acid residue of similar chemical structure (e.g., hydrophilicity, degree and distribution of charged regions). Such substitutions are recognized in the art as typically involving a minor change with no or essentially no influence on the function, activity or other biological properties of the polypeptide. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. In preferred embodiments, a conserved amino acid substitution involves substitution of amino acids having hydropathic indexes of ±2.

Within the context of this disclosure, each amino acid sequence described herein can also be interpreted as an amino acid sequence with a sequence identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence. In a preferred embodiment, sequence identity is determined by comparing the whole length of the sequences as identified herein.

Within the context of this disclosure, each amino acid sequence described herein can also be interpreted as containing one conservative substitution of a CDR sequence; or alternatively, one, two, three, four, five, six, or seven conservative amino acid sequence substitutions in non-CDR sequences.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant is also used herein to describe a protein with an amino acid sequence that is substantially identical to a referenced protein with an amino acid sequence that retains at least one biological activity. “Variant” also can be used to refer to an antigenically reactive fragment of an anti-MuSK antibody that differs from the corresponding fragment of anti-MuSK antibody in amino acid sequence but is still antigenically reactive and can compete with the corresponding fragment of anti-MuSK antibody for binding with MuSK. “Variant” also can be used to describe a polypeptide or a fragment thereof that has been differentially processed, such as by proteolysis, phosphorylation, or other post-translational modification, yet retains its antigen reactivity.

The term “vector,” as used herein, refers to a nucleic acid molecule capable of propagating a nucleic acid to which the vector sequence is linked, in a host cell in which the vector is introduced.

MuSK may have the following amino acid sequence:

(SEQ ID NO: 1)
MRELVNIPLV HILTLVAFSG TEKLPKAPVI TTPLETVDAL VEEVATFMCA VESYPQPEIS
WTRNKILIKL FDTRYSIREN GQLLTILSVE DSDDGIYCCT ANNGVGGAVE
SCGALQVKMK PKITRPPINV KIIEGLKAVL PCTTMGNPKP SVSWIKGDSP
LRENSRIAVL ESGSLRIHNV QKEDAGQYRC VAKNSLGTAY SKVVKLEVEV
FARILRAPES HNVTFGSFVT LHCTATGIPV PTITWIENGN AVSSGSIQES
VKDRVIDSRL QLFITKPGLY TCIATNKHGE KFSTAKAAAT ISIAEWSKPQ
KDNKGYCAQY RGEVCNAVLA KDALVFLNTS YADPEEAQEL LVHTAWNELK
VVSPVCRPAA EALLCNHIFQ ECSPGVVPTP IPICREYCLA VKELFCAKEW
LVMEEKTHRG LYRSEMHLLS VPECSKLPSM HWDPTACARL PHLDYNKENL
KTFPPMTSSK PSVDIPNLPS SSSSSFSVSP TYSMTVIISI MSSFAIFVLL TITTLYCCRR
RKQWKNKKRE SAAVTLTTLP SELLLDRLHP NPMYQRMPLL LNPKLLSLEY
PRNNIEYVRD IGEGAFGRVF QARAPGLLPY EPFTMVAVKM LKEEASADMQ
ADFQREAALM AEFDNPNIVK LLGVCAVGKP MCLLFEYMAY GDLNEFLRSM
SPHTVCSLSH SDLSMRAQVS SPGPPPLSCA EQLCIARQVA AGMAYLSERK
FVHRDLATRN CLVGENMVVK IADFGLSRNI YSADYYKANE NDAIPIRWMP
PESIFYNRYT TESDVWAYGV VLWEIFSYGL QPYYGMAHEE VIYYVRDGNI
LSCPENCPVE LYNLMRLCWS KLPADRPSFT SIHRILERMC ERAEGTVSV.

MuSK may be a variant or fragment of SEQ ID NO:1. Exemplary variants include D38E, P344R, 1575T, M6051, A727V, V790M and M835V. Alternate variants include deletion, insertion, or substitution of the amino acid at position 27, 38, 100, 107, 159, 222, 344, 413, 575, 584, 605, 609, 629, 644, 664, 696, 727, 743, 782, 790, 819, 829, 835, or 858 of SEQ ID NO:1. As used herein, amino acids at positions 28-116 of SEQ ID NO:1 is the “Ig-1 domain”; amino acids at positions 121-205 of SEQ ID NO:1 is the “Ig-2 domain”; amino acids at positions 212-302 of SEQ ID NO:1 is the “Ig-3 domain”; amino acids at positions 312-450 of SEQ ID NO:1 is the “frizzled-like cysteine-rich domain (FZ-CRD) domain”; and amino acids at positions 575-856 of SEQ ID NO:1 is the “Protein Kinase domain.”

In a first aspect, the present disclosure describes an anti-MuSK antibody capable of binding MuSK (SEQ ID NO:1) or a variant thereof (i.e., anti-MuSK agonistic antibody).

An anti-MuSK antibody of the present disclosure may comprise a variable heavy chain comprising a CDR1 with the amino acid sequence set forth in SEQ ID NOs: 3, 11, 19, 27, 35, 43, 51, 59, 67, 75, 83, 91, 99, 107, 115, 123, 131, 139, 147, 155, 163, or 171, a CDR2 with the amino acid sequence set forth in SEQ ID NOs: 4, 12, 20, 28, 36, 44, 52, 60, 68, 76, 84, 92, 100, 108, 116, 124, 132, 140, 148, 156, 164, or 172; and a CDR3 with the amino acid sequence set forth in SEQ ID NOs: 5, 13, 21, 29, 37, 45, 53, 61, 69, 77, 85, 93, 101, 109, 117, 125, 133, 141, 149, 157, 165, or 173.

An anti-MuSK antibody of the present disclosure may comprise a variable heavy chain comprising the amino acid sequence set forth in SEQ ID NOs: 2, 10, 18, 26, 34, 42, 50, 58, 66, 74, 82, 90, 98, 106, 114, 122, 130, 138, 146, 154, 162, or 170.

An anti-MuSK antibody of the present disclosure may comprise a variable light chain comprising a CDR1 with the amino acid sequence set forth in SEQ ID NOs: 7, 15, 23, 31, 39, 47, 55, 63, 71, 79, 87, 95, 103, 111, 119, 127, 135, 143, 151, 159, 167, or 175; a CDR2 with the amino acid sequence set forth in SEQ ID NOs: 8, 16, 24, 32, 40, 48, 56, 64, 72, 80, 88, 96, 104, 112, 120, 128, 136, 144, 152, 160, 168, or 176; and a CDR3 with the amino acid sequence set forth in SEQ ID NOs: 9, 17, 25, 33, 41, 49, 57, 65, 73, 81, 89, 97, 105, 113, 121, 129, 137, 145, 153, 161, 169, or 177.

An anti-MuSK antibody of the present disclosure may comprise a variable light chain comprising the amino acid sequence set forth in SEQ ID NOs: 6, 14, 22, 30, 38, 46, 54, 62, 70, 78, 86, 94, 102, 110, 118, 126, 134, 142, 150, 158, 166, or 174.

Exemplary anti-MuSK agonistic antibodies include 02E22A as defined in Table 1, 03F07A as defined in Table 2, 03P23A as defined in Table 3, 04F19A as defined in Table 4, 04H19B as defined in Table 5, 04N24A as defined in Table 6, 05F22A as defined in Table 7, 05K18A as defined in Table 8, 05L17A as defined in Table 9, 05M23A as defined in Table 10, 06M22A as defined in Table 11, 06022A as defined in Table 12, 07A08A as defined in Table 13, 07E07A as defined in Table 14, 08C23A as defined in Table 15, 08D04A as defined in Table 16, 09A16A as defined in Table 17, 09F14A as defined in Table 18, 09G09A as defined in Table 19, 10M10A as defined in Table 20, 11106A as defined in Table 21, and 12P23A as defined in Table 22.

TABLE 1

| SEQ ID | | |
|---|---|---|
| NO: | 02E22A | |
| 2 | VH | EVQLVESGGGLVQPGGSLRLSCAASGFIFSIYWMSWVR |
| | | QAPGKGLEWVANINQDGSEKYYVDSVKGRFTISRDNAK |
| | | NSLYLQMNSLRAEDTAVYYCAREVGTTDWGQGTLVTVS |
| | | S |
| 3 | HC CDR1 | GFIFSIYW |
| 4 | HC CDR2 | INQDGSEK |
| 5 | HC CDR3 | AREVGTTD |
| 6 | VL | NFMLTQPHSVSESPGKTVTISCTRSSGGIASNYVQWYQ |
| | | QRPGSSPTTVIYEDNQRPSGVPDRFSGSIDSSSNSASLTI |
| | | SGLKTEDEADYYCQSYDSSNWVLGGGTKLTVL |
| 7 | LC CDR1 | SGGIASNY |
| 8 | LC CDR2 | EDN |
| 9 | LC CDR3 | QSYDSSNWV |

TABLE 2

| SEQ ID | | |
|---|---|---|
| NO: | 03F07A | |
| 10 | VH | QVQLQESGPGLVKPSETLSLTCTVSGGSISSYYWSWIRQP |
| | | PGKGLEWIGYIYFSGRTYYNPSLKSRVTISVDTSKNQFSLK |
| | | LSSVTAADTAVYYCARGGGLGISNWFDPWGQGTLVTVSS |
| 11 | HC CDR1 | GGSISSYY |
| 12 | HC CDR2 | IYFSGRT |
| 13 | HC CDR3 | ARGGGLGISNWFDP |
| 14 | VL | SYELTQPPSVSVSPGQTARITCSGDALPKQYAYWYQQKP |
| | | GQAPVLVIYKDSERPSGIPERFSGSSSGTTVTLTISGVQAE |
| | | DEADYYCQSADSSGTYWVFGGGTKLTVL |
| 15 | LC CDR1 | ALPKQY |
| 16 | LC CDR2 | KDS |
| 17 | LC CDR3 | QSADSSGTYWV |

TABLE 3

| SEQ ID | | |
|---|---|---|
| NO: | 03P23A | |
| 18 | VH | QVQLVQSGAEVKKPGSSVKVSCKASGGTFSSYGISWVRQ |
| | | APGQGLEWMGRIIPIFGTANYAQKFQGRVTITADKSTSTAY |
| | | MELSSLRSEDTAVYYCARSRFLEWLLNYYGMDVWGQGTT |
| | | VTVSS |
| 19 | HC CDR1 | GGTFSSYG |
| 20 | HC CDR2 | IIPIFGTA |
| 21 | HC CDR3 | ARSRFLEWLLNYYGMDV |
| 22 | VL | DIVMTQSPLSLPVTPGEPASISCRSSQSLLHSNGYNYLDW |
| | | YLQKPGQSPQFLIYLGSNRASGVPDRFSGSGSGTDFTLKI |
| | | SRVEAEDVGVYYCMQTLQTPPTFGQGTKVEIK |
| 23 | LC CDR1 | QSLLHSNGYNY |
| 24 | LC CDR2 | LGS |
| 25 | LC CDR3 | MQTLQTPPT |

TABLE 4

| SEQ ID | | |
|---|---|---|
| NO: | 04F19A | |
| 26 | VH | QVQLVQSGAEVKKPGASVKVSCKASGYNFAGYYMHWVR |
| | | QAPGQGLEWMGWISPSSGGTNYAQKFQGRVTMTRDTSI |
| | | STAYMELSRLRSDDTAVYYCASGGVVVVAAFDYWGQGTL |
| | | VTVSS |
| 27 | HC CDR1 | GYNFAGYY |
| 28 | HC CDR2 | ISPSSGGT |
| 29 | HC CDR3 | ASGGVVVVAAFDY |
| 30 | VL | QSVLTQPPSVSGAPGQRVTISCTGSSSNIGTGYDVHWYQ |
| | | QLPGTAPKLLIYNHSHRPSGVPDRFSGSKSGTSASLAITGL |
| | | QAEDEADYYCQSYDSSLSGSWVFGGGTKLTVL |
| 31 | LC CDR1 | SSNIGTGYD |
| 32 | LC CDR2 | NHS |
| 33 | LC CDR3 | QSYDSSLSGSWV |

TABLE 5

| SEQ ID | | |
|---|---|---|
| NO: | 04H19B | |
| 34 | VH | EVQLVQSGAEVKKPGESLKISCKGSGYSFTSYWIGWVRQ |
| | | MPGKGLEWMGIIYPGDSDTRYSPSFQGQVTISADKSISTA |
| | | YLQWSSLKASDTAMYYCARHSHWGFLDYWGQGTLVTVS |
| | | S |
| 35 | HC CDR1 | GYSFTSYW |
| 36 | HC CDR2 | IYPGDSDT |
| 37 | HC CDR3 | ARHSHWGFLDY |
| 38 | VL | DIVMTQSPLSLPVIPGEPASISCRSSQSLLQSNGYNYLDWY |
| | | LQKPGQSPQLLIYLGSNRASGVPDRFSGSGSGTDFTLKIS |
| | | RVEAEDVGVYYCMQVQQTPYTFGQGTKLEIK |
| 39 | LC CDR1 | QSLLQSNGYNY |
| 40 | LC CDR2 | LGS |
| 41 | LC CDR3 | MQVQQTPYT |

TABLE 6

| SEQ ID | | |
|---|---|---|
| NO: | 04N24A | |
| 42 | VH | QVQLQESGPGLVKPSETLSLTCTVSGGSISSYYWTWIRQP |
| | | AGKGLEWIGRIYTSGSTDYNPSLKSRVTMSVDTSKNQFSL |
| | | KLSSVTAADTAVYYCARYNWNDDAFDIWGQGTMVTVSS |
| 43 | HC CDR1 | GGSISSYY |
| 44 | HC CDR2 | IYTSGST |
| 45 | HC CDR3 | ARYNWNDDAFDI |
| 46 | VL | DIVMTQSPLSLPVTPGEPASISCRSSQSLLHRNGYNYLDW |
| | | YLQKPGQSPQLLIYLGSNRASGVPDRFSGSGSGTDFTLKI |
| | | SRVEAEDVGIYYCMQALQIPHTFGQGTKLEIK |
| 47 | LC CDR1 | QSLLHRNGYNY |
| 48 | LC CDR2 | LGS |
| 49 | LC CDR3 | MQALQIPHT |

TABLE 7

| SEQ ID | | |
|---|---|---|
| NO: | 05F22A | |
| 50 | VH | QVQLQESGPGLVKPSQTLSLTCTVSGGSISSGGYCWSWI |
| | | RQHPGKGLEWIGYIYYSGSTYYNPSLKSRVTISEDTSKNQ |
| | | FSLKLSSVTAADTAVYYCARGGRWLQLRDVFDIWGQGTM |
| | | VTVSS |
| 51 | HC CDR1 | GGSISSGGYC |
| 52 | HC CDR2 | IYYSGST |
| 53 | HC CDR3 | ARGGRWLQLRDVFDI |
| 54 | VL | EIVLTQSPGTLSLSPGERATLSCRASQSVSSSYLAWYQQK |
| | | PGQAPRLLIYGASSRATGIPDRFSGSGSGTDFTLTISRLEP |
| | | EDFAAYHCQQHSSSPLTFGGGTKVEIK |
| 55 | LC CDR1 | QSVSSSY |
| 56 | LC CDR2 | GAS |
| 57 | LC CDR3 | QQHSSSPLT |

TABLE 8

| SEQ ID | | |
|---|---|---|
| NO: | 05K18A | |
| 58 | VH | QVQLQESGPGLVKPSESLSLTCTVSGDSISSYYWSWIRQP |
| | | AGKGLEWIGRFYTSGRSNYNPSLKSRVTMAVDTSKNQFS |
| | | LKLSSVTAADTAVYYCARDGFGSYFGSFDYWGQGTLVTV |
| | | SS |
| 59 | HC CDR1 | GDSISSYY |
| 60 | HC CDR2 | FYTSGRS |
| 61 | HC CDR3 | ARDGFGSYFGSFDY |
| 62 | VL | EIVMTQSPATLSVSPGERATLSCRASQSIISNLAWYQQKP |
| | | GQAPRLLIYDASTRAAGIPARFSGSGSGTEFTLTISTLQSE |
| | | DFAVYYCQQYNKWPTFGGGTKVEIK |
| 63 | LC CDR1 | QSIISN |
| 64 | LC CDR2 | DAS |
| 65 | LC CDR3 | QQYNKWPT |

TABLE 9

| SEQ ID | | |
|---|---|---|
| NO: | 05L17A | |
| 66 | VH | EVQLVESGGGLVKPGGSLRLSCAASGFTFSSYSMNWVRQA |
| | | PGKGLEWVSSISSSSSYIYYTDSVKGRFTISRDNAKNSLYLQ |
| | | MNSLSAEDTAVYYCARDRYDRSGYYYVPFDQWGQGTLVT |
| | | VSS |
| 67 | HC | GFTFSSYS |
| | CDR1 | |
| 68 | HC | ISSSSSYI |
| | CDR2 | |
| 69 | HC | ARDRYDRSGYYYVPFDQ |
| | CDR3 | |
| 70 | VL | DIQMTQSPSTLSASVGDRVTITCRASQSITSWLAWYQQKPG |
| | | KAPKLLIYKASSLESGVPSRFSGSGSGTEFTLTISSLQPDDF |
| | | ATYYCQQYNSFSWTFGQGTKVEIK |
| 71 | LC | QSITSW |
| | CDR1 | |
| 72 | LC | KAS |
| | CDR2 | |
| 73 | LC | QQYNSFSWT |
| | CDR3 | |

TABLE 10

| SEQ ID | | |
|---|---|---|
| NO: | 05M23A | |
| 74 | VH | QVQLHESGPGLVKPSETLSLTCTVSGGSISSYYWSWIRQPA |
| | | GKGVEWIGRIYPSGSTNYNPSLKSRVIMSVDTSKSQFSLKLS |
| | | SVTAADTAVYYCARYSGICYGAFDIWGQGTMVTVSS |
| 75 | HC | GGSISSYY |
| | CDR1 | |
| 76 | HC | IYPSGST |
| | CDR2 | |
| 77 | HC | ARYSGICYGAFDI |
| | CDR3 | |
| 78 | VL | EIVLTQSPGTLSLSPGERATLSCRASQSVSSSYLAWYQQKP |
| | | GQAPRLLIYGASSRATGIPDRFSGSGSGTEFTLTISRLEPED |
| | | FAVYYCQQHGSSPRTFGQGTKVEIK |
| 79 | LC | QSVSSSY |
| | CDR1 | |
| 80 | LC | GAS |
| | CDR2 | |
| 81 | LC | QQHGSSPRT |
| | CDR3 | |

TABLE 11

| SEQ ID | | |
|---|---|---|
| NO: | 06M22A | |
| 82 | VH | QVQLQESGPGLVKPSETLSLTCTVSGDSISSYFWTWIRQPA |
| | | GKGLEWIGRIYTSGITNYNPSLKSRVTMSVDTSKNQFSLKLN |
| | | SVTAADTAVYYCARDTSSVGEFDYWGQGTLVTVSS |
| 83 | HC | GDSISSYF |
| | CDR1 | |
| 84 | HC | IYTSGIT |
| | CDR2 | |
| 85 | HC | ARDTSSVGEFDY |
| | CDR3 | |
| 86 | VL | QSALTQPPSASGSPGQSVTISCTGTSSDVGGYNYVSWYQQ |
| | | HPGKAPKLMIYEVSKRPSGVPDRFSGSKSGNTASLTVSGLQ |
| | | AEDEADYYCSSYAGSNNLVFGGGTKLTVL |
| 87 | LC | SSDVGGYNY |
| | CDR1 | |
| 88 | LC | EVS |
| | CDR2 | |
| 89 | LC | SSYAGSNNLV |
| | CDR3 | |

TABLE 12

| SEQ ID | | |
|---|---|---|
| NO: | 06022A | |
| 90 | VH | QVQLVQSGAEVKKPGASVKVSCKASGYTFTSYYMHWVRQ |
| | | APGQGLEWMGIINPSGGSTSYAQKFQGRVSMTSDTSTSTV |
| | | SMELSSLRSEDTAIYYCARELGWGSDYWGQGTLVTVSS |
| 91 | HC | GYTFTSYY |
| | CDR1 | |
| 92 | HC | INPSGGST |
| | CDR2 | |
| 93 | HC | ARELGWGSDY |
| | CDR3 | |
| 94 | VL | DIQMTQSPSAMSASVGDRVTITCRASQDIRNYLAWFQQKPG |
| | | KVPKRLIYIASNLQSGVPSRFSGSGSGTEFTLTISSLQPEDFA |
| | | TYYCLQHNSYPYTFGQGTKLEIK |
| 95 | LC | QDIRNY |
| | CDR1 | |
| 96 | LC | IAS |
| | CDR2 | |
| 97 | LC | LQHNSYPYT |
| | CDR3 | |

TABLE 13

| SEQ ID | | |
|---|---|---|
| NO: | 07A08A | |
| 98 | VH | QVQLQESGLGLVKPSETLSLTCTVSGGSISSYYWSWIRQPP |
| | | GKGLEWIGYIYSSGSTNDNPSLKSRVTISVDTSKNQFSLRLS |
| | | SVTVADTAVYYCARFNWGNDAFDIWGQGTMVTVSS |
| 99 | HC | GGSISSYY |
| | CDR1 | |
| 100 | HC | IYSSGST |
| | CDR2 | |
| 101 | HC | ARFNWGNDAFDI |
| | CDR3 | |
| 102 | VL | DIVMTQSPLSLPVTPGEPASISCRSSQSLLHRNGYNYLDWY |
| | | LQKPGQSPQLLIYLGSNRASGVTDRFSGSGSGTDFTLKISR |
| | | VEAEDVGVYYCMQALQTPFTFGPGTKVDIK |
| 103 | LC | QSLLHRNGYNY |
| | CDR1 | |
| 104 | LC | LGS |
| | CDR2 | |
| 105 | LC | MQALQTPFT |
| | CDR3 | |

TABLE 14

| SEQ ID | | |
|---|---|---|
| NO: | 07E07A | |
| 106 | VH | QVQLVQSGAEEKKPGASVKVSCKASGYTFTNYYMHWVRQ |
| | | APGQGLEWMGIINPSGGNTSYAQKFQGRVTMTRDTSTSTV |
| | | YMELSSLRSEDTAVYYCARELGWGSDYWGQGTLVTVSS |
| 107 | HC | GYTFTNYY |
| | CDR1 | |
| 108 | HC | INPSGGNT |
| | CDR2 | |
| 109 | HC | ARELGWGSDY |
| | CDR3 | |
| 110 | VL | DIQMTQSPSAMSASIGDRVTITCRASQDISNYLAWFQQKPG |
| | | KVPKRLIYVASGLQSGVPSRFSGSGSGTEFTLTISSLQPEDF |
| | | ATYYCLQHNSYPFTFGQGTKLEIK |
| 111 | LC | QDISNY |
| | CDR1 | |
| 112 | LC | VAS |
| | CDR2 | |
| 113 | LC | LQHNSYPFT |
| | CDR3 | |

TABLE 15

| SEQ ID | | |
|---|---|---|
| NO: | 08C23A | |
| 114 | VH | QVQLQESGPGLVKPSETLSLTCTVSSGSISPYYWSWIRQPP |
| | | GKGLEWIGYIYYRGTTNYNPSLQSRVTMSVDTSKNQFSLKL |
| | | SSVTAADTAVYYCARAALGAVFDYWGQGTLVTVSS |
| 115 | HC | SGSISPYY |
| | CDR1 | |
| 116 | HC | IYYRGTT |
| | CDR2 | |
| 117 | HC | ARAALGAVFDY |
| | CDR3 | |
| 118 | VL | QSVLTQPPSVSAAPGQKVTISCSGSSSNIGNNYVSWYQQLP |
| | | GTAPKLLIYDNNKRPSGIPDRFSGSKSGTSATLGITGLQTGD |
| | | EADYYCGTWDSSLSAVFFGGGTNLTVL |
| 119 | LC | SSNIGNNY |
| | CDR1 | |
| 120 | LC | DNN |
| | CDR2 | |
| 121 | LC | GTWDSSLSAVF |
| | CDR3 | |

TABLE 16

| SEQ ID | | |
|---|---|---|
| NO: | 08D04A | |
| 122 | VH | QVQLVQSGAEVKKPGASVKVSCKASGYTFTSYYIHWVRQA |
| | | PGQGLEWMGIINPSGGSISYAQKFQGRVTLTRDTSTSIVYM |
| | | ELSSLRSEDTAVYYCARGGTYYYYHGMDVWGQGTTVTVSS |
| 123 | HC | GYTFTSYY |
| | CDR1 | |
| 124 | HC | INPSGGSI |
| | CDR2 | |
| 125 | HC | ARGGTYYYYHGMDV |
| | CDR3 | |
| 126 | VL | QSALTQPASVSGSPGQSITISCTGTSSDVGSYNLVSWYQQH |
| | | PGKAPKLMIYEGSKRPSGVSNRFSGSKSGNTASLTISGLQA |
| | | EDEADYYCCSYAGSSTWVFGGGTKLTVL |
| 127 | LC | SSDVGSYNL |
| | CDR1 | |
| 128 | LC | EGS |
| | CDR2 | |
| 129 | LC | CSYAGSSTWV |
| | CDR3 | |

TABLE 17

| SEQ ID | | |
|---|---|---|
| NO: | 09A16A | |
| 130 | VH | EVQLVQSGAEVKKPGESLKISCKGSGYSFISHWIGWVRQMP |
| | | GKGLEWMGIIYPGDSDTRYSPSFQGQVTISADKSISTAYLQ |
| | | WSSLKASDTAIYYCARYYYYYGMDVWGQGTTVTVSS |
| 131 | HC | GYSFISHW |
| | CDR1 | |
| 132 | HC | IYPGDSDT |
| | CDR2 | |
| 133 | HC | ARYYYYYGMDV |
| | CDR3 | |
| 134 | VL | EIVMTQSPVTLSVSPGERATLSCRASQSVSSNLAWYQQKP |
| | | GQAPRFLIYGASTRTTGIPARFSGSGSGTEFTLTISSLQSED |
| | | FAVYYCQQYNNWPYTFGQGTKLEIK |
| 135 | LC | QSVSSN |
| | CDR1 | |
| 136 | LC | GAS |
| | CDR2 | |
| 137 | LC | QQYNNWPYT |
| | CDR3 | |

TABLE 18

| SEQ ID | | |
|---|---|---|
| NO: | 09F14A | |
| 138 | VH | QVQLQESGPGLVKPSQTLSLTCTVSGGSISSGGHYWNWIR |
| | | QHPGKGLEWIGYIYYSGTTYYNPSLKSRVTISVDTSKNQFSL |
| | | KLSSVTAADTAVYYCAREVGIAFDYWGQGTLVTVSS |
| 139 | HC | GGSISSGGHY |
| | CDR1 | |
| 140 | HC | IYYSGTT |
| | CDR2 | |
| 141 | HC | AREVGIAFDY |
| | CDR3 | |
| 142 | VL | DIQLTQSPSFLSASVGDRVTITCRASQGVSSYLAWYQQKPG |
| | | KAPKLLIFAASALQSGVPSRFSGSGSGTEFTLTISSLQPEDF |
| | | ATYHCQQLNSYPPTFGQGTKVEIK |
| 143 | LC | QGVSSY |
| | CDR1 | |
| 144 | LC | AAS |
| | CDR2 | |
| 145 | LC | QQLNSYPPT |
| | CDR3 | |

TABLE 19

| SEQ ID | | |
|---|---|---|
| NO: | 09G09A | |
| 146 | VH | EVQLVQSGAEVKKPGESLKISCKGSGYNFPNYWIAWVRQM |
| | | PGKGLEWMGIIYPGDSDTRYSPSFQGQVTISADKSISTAYLQ |
| | | WSSLKASDTAMYYCARHSNWGHFDYWGQGTLVTVSS |
| 147 | HC | GYNFPNYW |
| | CDR1 | |
| 148 | HC | IYPGDSDT |
| | CDR2 | |
| 149 | HC | ARHSNWGHFDY |
| | CDR3 | |
| 150 | VL | DIVMTQSPLSLPVTPGEPASISCRSSQSLLHSNGYNYLDWY |
| | | LQKPGQSPQVLIYMGSNRASGVPDRFSGSGSGTDFTLKISR |
| | | VEAEDVGVYYCMQAQQTPYTFGQGTKLEIK |
| 151 | LC | QSLLHSNGYNY |
| | CDR1 | |
| 152 | LC | MGS |
| | CDR2 | |
| 153 | LC | MQAQQTPYT |
| | CDR3 | |

TABLE 20

| SEQ ID | | |
|---|---|---|
| NO: | 10M10A | |
| 154 | VH | EVQLVESGGGLVQPGGSLRLSCAASGFTFSSYWMSWARQ |
| | | APGKGLEWVANIKQDGSEKYYVDSVKGRFTISRDNAKNSLY |
| | | LQMNSLRVEDTAVYYCASSGIVGASTLDYWGQGILLIVSA |
| 155 | HC | GFTFSSYW |
| | CDR1 | |
| 156 | HC | IKQDGSEK |
| | CDR2 | |
| 157 | HC | ASSGIVGASTLDY |
| | CDR3 | |
| 158 | VL | EIVMTQSPATMSVSPGERATLSCRASQSVSSNLAWFQQKP |
| | | GQAPRLLIYGASTRATGIPARFSGSGSGTEFTLTISSLQSED |
| | | FAVYYCQQYNNWPPYTFGQGTKLEIK |
| 159 | LC | QSVSSN |
| | CDR1 | |
| 160 | LC | GAS |
| | CDR2 | |
| 161 | LC | QQYNNWPPYT |
| | CDR3 | |

TABLE 21

| SEQ ID | | |
|---|---|---|
| NO: | 11106A | |
| 162 | VH | EVQLVESGGGLVQPGGSLRLSCAASGFTFSSYRLNWVRQA |
| | | PGKGLEWVSYISSSTGSLDYADSVKGRFTISRDNAKNSLYL |
| | | QMNSLRDEDTAVYYCARRSPYTGSLDNWGQGTLVTVSS |
| 163 | HC | GFTFSSYR |
| | CDR1 | |
| 164 | HC | ISSSTGSL |
| | CDR2 | |
| 165 | HC | ARRSPYTGSLDN |
| | CDR3 | |
| 166 | VL | DIVMTQSPLSLPVTPGEPASISCRSSQSLLHSNGYNYLDWY |
| | | LQKPGQSPQLLIYLGSNRASGVPDRFSGSGSGTDFTLKINR |
| | | VEAEDVGVYYCMQALQTPYTFGQGTKLEIK |
| 167 | LC | QSLLHSNGYNY |
| | CDR1 | |
| 168 | LC | LGS |
| | CDR2 | |
| 169 | LC | MQALQTPYT |
| | CDR3 | |

TABLE 22

| SEQ ID | | |
|---|---|---|
| NO: | 12P23A | |
| 170 | VH | QVQLVQSGAEVKKPGSSVKVSCKASGGTFSSYGISWVRQA |
| | | PGQGLEWMGGIIPFFGSANYAQKFQGRVTITADKSTSTAYM |
| | | ELSSLRSEDTAVYYCARDGGAILDYWGQGTLVTVSS |
| 171 | HC | GGTFSSYG |
| | CDR1 | |
| 172 | HC | IIPFFGSA |
| | CDR2 | |
| 173 | HC | ARDGGAILDY |
| | CDR3 | |
| 174 | VL | SYELTQPSSVSVSPGQTARITCSGDVLAKKYARWFQQKPG |
| | | QAPLLVIYKDSERPSGIPERFSGSSSGTTVTLTISGAQVEDE |
| | | ADYYCYSAADNNWVFGGGTKLTVL |
| 175 | LC | VLAKKY |
| | CDR1 | |
| 176 | LC | KDS |
| | CDR2 | |
| 177 | LC | YSAADNNWV |
| | CDR3 | |

An anti-MuSK antibody disclosed herein may be a monoclonal antibody, a humanized antibody, a chimeric antibody, a single chain antibody, a Fab fragment, a single-chain variable fragment (scFv), a recombinant antibody, a recombinant monoclonal antibody, an aptamer, a single-domain antibody (VHH, nanobody), or other MuSK-binding fragment or variant. In certain embodiments, an anti-MuSK antibody disclosed herein may comprise complementarity determining regions (CDRs) from a source (parental) antibody that have been grafted (fused) into a framework from a different type (class) of antibody and/or from a different organism than the parental antibody, in particular an acceptor human framework. In certain embodiments, an anti-MuSK antibody disclosed herein may comprise a framework in which amino acids have been substituted, mutated, or replaced in regions outside of the CDRs to influence properties such as antigen-binding or antibody structure, e.g., in the variable region framework surrounding the CDRs and/or in a constant region, in particular the Fc region. In certain embodiments, one or more of the CDRs have been substituted, mutated, or replaced. In certain embodiments, an anti-MuSK antibody disclosed herein may be a humanized anti-MuSK antibody variant. In preferred embodiments, the anti-MuSK antibody is a monoclonal antibody. In other preferred embodiments, the anti-MuSK antibody is a human or fully human monoclonal antibody.

In a second aspect, the present disclosure describes an anti-MuSK agonistic antibody capable of activating the signaling and/or phosphorylation of MuSK. In embodiments, the β-subunits of MuSK are phosphorylated, preferably, tyrosine resides of the β-subunits of MuSK.

In embodiments, the anti-MusK agonistic antibody is capable of binding an epitope in the FZ-CRD domain of MuSK. Exemplary antibodies include 03F07A, 03P23A, 04F19A, 04H19B, 04N24A, 05F22A, 05K18A, 05L17A, 05M23A, 06M22A, 06022A, 07A08A, 07E07A, 08C23A, 09A16A, 09F14A, 09G09A, 10M10A, 11106A, and 12P23A. Preferred anti-MuSK agonistic antibodies include 02E22A, 07A08A, 09A16A, 09F14A, 09G09A, and 12P23A.

In other embodiments, the anti-MusK agonistic antibody is capable of binding an epitope in the Ig-3 domain of MuSK. Exemplary antibodies include 02E22A and 08D04A.

Without being bound by a particular theory, the inventors have found that binding of the FZ-CRD domain or Ig-3 domain is associated with the desired effect of activating MuSK while not frustrating the ability of MuSK to interact with other binding proteins.

In a third aspect, the present disclosure describes an anti-MuSK agonistic antibody useful to restore and/or increase MuSK function. In embodiments, use of the anti-MuSK antibody is associated with the enlargement of nerve terminals at NMJs. In other embodiments, use of the anti-MuSK antibody is associated with reversal of denervation at NMJs.

In a fourth aspect, the present disclosure describes an anti-MuSK agonistic antibody useful to increase AChR clustering.

In a fifth aspect, the present disclosure describes an anti-MuSK agonistic antibody useful to counteract natural autoantibodies that target and block the function of MuSK. Such antibodies may be IgG antibodies (e.g., of the IgG1, IgG2, IgG3, or, preferably, IgG4 subclass).

In a sixth aspect, the present disclosure describes a nucleic acid comprising a nucleotide sequence encoding an anti-MuSK antibody disclosed herein.

In a seventh aspect, the present disclosure describes a host cell, such as a eukaryotic cell (e.g., a mammalian host cell), that is transformed with a vector comprising a nucleic acid comprising a nucleotide sequence encoding an anti-MuSK antibody disclosed herein.

In an eighth aspect, the present disclosure describes a method of producing an anti-MuSK antibody disclosed herein comprising culturing a host cell and recovering the anti-MuSK antibody.

### EXAMPLES

Example 1. Generation and Identification of Anti-MuSK antibodies AlivaMab Mice, a transgenic mouse line producing antibodies with fully-human F(ab′)₂ and mouse CH2 and CH3 domains wherein the fully human F(ab′)₂ comprises human VH and VL, and human CL and CH1, was utilized from Ablexis, LLC (San Diego, CA) and used in immunization and generation of antibodies of the present disclosure. Nine Kappa-Lambda AlivaMab Mice (3× AMM-XKL1, 3× XKL2, and 3× XKL3) (from AlivaMab Biologics, LLC (formerly AlivaMab Discovery Services, LLC, San Diego, CA)) were primed with a mixture of DNA expressing full-length human MuSK conjugated with super T cell epitope (TCE) (pADS38-huMuSK FL-TCE) and DNA expressing full-length mouse MuSK conjugated with TCE (pADS38-muMuSK FL-TCE). The mice were then boosted 6 times with a mixture of recombinant proteins and recombinant proteins conjugated with TCE, including huMuSK Ig3-Fz (recombinant protein containing Ig3 and Fz domains of human MuSK), muMuSK Ig3-Fz (recombinant protein containing Ig3 and Fz domains of mouse MuSK), huMuSK Ig3-Fz-TCE conjugate, and muMuSK Ig3-Fz-TCE conjugate. Before the final boost, plasma titers were checked to ensure they were sufficient. Plasma titers were determined by fluorescence-activated cell sorting (FACS) against HEK293T cell lines stably expressing human, mouse, or cynomolgus monkey MuSK (293T: huMuSK, 293T: muMuSK, or 293T: cyMuSK) and by enzyme-linked immunosorbent assay (ELISA) against recombinant protein containing the full extracellular domain (ECD) of human or mouse MuSK (huMuSK ECD or muMuSK ECD). Following the final boost, B cells were isolated and enriched from spleens and lymph nodes, and subsequently used for electrofusion to generate hybridomas.

Hybridomas were plated into 384-well plates at a density of 1-2 hybridomas per well. Hybridoma supernatants were screened by FACS against 293T: huMuSK, 293T: muMuSK, or parental 293T and by ELISA against huMuSK Ig3-Fz or muMuSK Ig3-Fz. 376 hybridomas were selected based on the FACS and ELISA results. Some of those hybridomas showed strong binding against 293T: huMuSK and 293T: muMuSK in FACS and strong binding against huMuSK Ig3-Fz and muMuSK Ig3-Fz in ELISA. Other hybridomas only showed strong binding against 293T: huMuSK and 293T: muMuSK in FACS.

The 376 hybridomas were then moved forward to a confirmation screen, which includes FACS against 293T: huMuSK, 293T: muMuSK, or 293T: cyMuSK and ELISA against huMuSK ECD or muMuSK ECD. 221 hybridomas were selected in the confirmation screen. 215 of them had high affinity with human, mouse, and cynomolgus monkey MuSK in FACS (hu/mu/cy MuSK triple binders). 6 of them had high affinity with human and cynomolgus monkey MuSK but low affinity with mouse MuSK in FACS and ELISA (hu/cy MuSK double binders). The selected hybridomas were then expanded. The saturated supernatants (supes) from the hybridomas were harvested for functional assays to identify MuSK agonist hybridomas.

Example 2. Identification of MuSK agonist hybridomas. A cell-based activation assay was developed for large-scale screening of MuSK agonist antibodies. A Ba/F3 cell pool stably expressing a chimeric receptor containing extracellular domain of human MuSK and transmembrane and intracellular domains of human Thrombopoietin receptor (c-MpI) was generated (Ba/F3: MuSK-cMpI). The activation of this chimeric receptor by MuSK agonist antibody causes the dimerization of the receptor activating the signaling pathway downstream of c-MpI and induces cell proliferation, which can be measured by proliferation assay. The saturated supes from 215 hybridomas selected from confirmation screen (hu/mu/cy MuSK triple binders), a reference monoclonal antibody activating MuSK (rfmAb-1) (which is characterized as the X17 antibody in Oury, J., et al. (2021)), Nature, 595 (7867): 404-408), and saturated supes containing antibodies not targeting MuSK (Negative Control) were tested for MuSK activation. Ba/F3: MuSK-cMpI cells were maintained in the culture medium containing RPMI-1640 (Gibco, 11875093), 10% FBS (Gibco, 10082-147), 1% Pen Strep (Gibco, 15140-122) and 5 ng/ml mouse IL-3 (Sino Biological, 51066-MNAH) in a CO2 incubator (5% CO2) at 37° C. Cells were washed once with RPMI-1640 and transferred to IL-3 free culture medium containing RPMI-1640, 10% FBS, and 1% anti-anti and incubated for 1 day. Cells were washed once with RPMI-1640, and treated for 3 days with 2% saturated supe, 2% spent medium or 1.5 μg/mL rfmAb-1 in IL-3 free medium in a 96-well culture plate (5000 cells in 100 μL medium per well). At the end of the treatment, cell proliferation was measured using CellTiter-Glo® Luminescent Cell Viability Assay kit (Promega, G7571) according to the manufacturer's instruction. Luminescence was measured in CLARIOstar® Plus Multi-mode Plate Reader (BMG Labtech). The Relative Light Unit (RLU) of all treated cells is listed in Table 1. The values are the average RLU of two wells under the same treatment. The average RLU of Negative Controls 1, 2 and 3 was around 11,000. The average RLU of rfmAb-1 treated cells was 176,506, indicating that rfmAb-1 induced activation of MuSK-cMpI receptor and proliferation of Ba/F3: MuSK-cMpI cells. Among the 215 hybridomas, 24 of them induced strong proliferation with the average RLU higher than 900,000. Luminescence intensity in the cell viability assay is reported in Table 23.

TABLE 23
Average RLU in cell viability assay of Ba/F3:
MuSK-cMpl cells treated with 2%
saturated supes or 1.5 μg/mL rfmAb-1 for 3 days.

| | Average | | Average |
|---|---|---|---|
| ID | RLU | ID | RLU |
| 09G09 | 2002594 | 03F07 | 1159873 |
| 04N24 | 1680767 | 04H19 | 1157780 |
| 07A08 | 1617142 | 06M22 | 1131016 |
| 10M10 | 1409447 | 12P23 | 1063935 |
| 09A16 | 1391862 | 04F19 | 1050781 |
| 05H24 | 1386905 | 06022 | 1028382 |
| 02E22 | 1292679 | 08C23 | 1016566 |
| 03P23 | 1262229 | 05F22 | 1003944 |
| 09F14 | 1262105 | 05K18 | 962991 |
| 07E07 | 1213657 | 05M23 | 901954 |
| 05L17 | 1210581 | rfmAb-1 | 176506 |
| 05G12 | 1198499 | Negative | 10177 |
| | | Control-1 | |
| 08D04 | 1193900 | Negative | 11462 |
| | | Control-2 | |
| 11106 | 1193287 | Negative | 11759 |
| | | Control-3 | |

Example 3. Characterization of Anti-MuSK agonist antibodies. Twenty-two (22) monoclonal antibodies (mAbs) were successfully subcloned and purified from the 24 hybridoma hits. The mAbs were in human F(ab′)2/mouse CH2-CH3 format, and their variable regions were sequenced. See, Tables 1-22.

The 22 mAbs were first tested in the MuSK activation assay using Ba/F3: MuSK-cMpI cells to verify their agonist activity. 1.5 μg/mL of mAbs was added to cells to induce cell proliferation. The luminescence intensity of the treated cells was listed in Table 24. All 22 mAbs and the reference antibody rfmAb-1 induced strong proliferation of Ba/F3: MuSK-cMpI cells, indicating their capability to activate MuSK. Several of mAbs exhibited particularly surprisingly high activation with 75× higher than the negative control and/or 5× the rfmAb-1 control.

TABLE 24
Average RLU in cell viability assay of Ba/F3:
MuSK-cMpl cells treated with
1.5 μg/mL of monoclonal antibodies for 3 days.

| | Average | Fold of |
|---|---|---|
| ID | RLU | control |
| 02E22A | 1217593 | 85 |
| 03F07A | 1209481 | 84 |
| 03P23A | 1013968 | 71 |
| 04F19A | 1070230 | 75 |
| 04H19B | 1326737 | 92 |
| 04N24A | 1379598 | 96 |
| 05F22A | 271679 | 19 |
| 05K18A | 981349 | 68 |
| 05L17A | 240542 | 17 |
| 05M23A | 710796 | 49 |
| 06M22A | 721103 | 50 |
| 06022A | 752074 | 52 |
| 07A08A | 1055191 | 73 |
| 07E07A | 768754 | 54 |
| 08C23A | 963522 | 67 |
| 08D04A | 658431 | 46 |
| 09A16A | 765939 | 53 |
| 09F14A | 889695 | 62 |
| 09G09A | 1150933 | 80 |
| 10M10A | 743196 | 52 |
| 11106A | 1122886 | 78 |
| 12P23A | 1124524 | 78 |
| rfmAb-1 | 201084 | 14 |
| Negative | 14363 | 1 |
| Control | | |

Next, the same twenty-two (22) mAbs were subjected to FACS KD analysis. Serial dilution of mAb (initial concentration 10 nM, 5-fold dilutions, 8-point dilution series in duplicate) were added to 293T: huMuSK, 293T: cyMuSK, or 293T: muMuSK cells seeded in 96-well plates (50,000 cells per well). Cells were incubated at 4° C. overnight. After washing with FACS buffer twice, cells were incubated with Goat anti-Mouse IgG (H+L) Cross-Adsorbed Secondary Antibody, Alexa Fluor™ 647 (Thermo Fisher, A32728) diluted at 1:2000 and Helix Blue diluted at 1:2000 at 4° C. for 30 min. After washing with FACS buffer twice, cells were subjected to iQue3 Screener for FACS analysis. Geometric mean fluorescence intensity (gMFI) of A647 was plotted in Graphpad Prism for EC50 calculation. The results of FACS analysis were listed in Table 25.

TABLE 25
FACS KD of 22 anti-MuSK mAbs against 293T:
huMuSK, 293T: cyMuSK, and 293T: muMuSK

| | 293T: huMuSK | | | 293T: cyMuSK | | | 293T: muMuSK | | |
|---|---|---|---|---|---|---|---|---|---|
| | | EC50 | | | EC50 | | | EC50 | |
| ID | gMFI max | (nM) | R² | gMFI max | (nM) | R² | gMFI max | (nM) | R² |
| 02E22A | 1837934 | 0.22 | 0.99 | 1555868 | 0.38 | 0.99 | 2082131 | 0.42 | 1.00 |
| 03F07A | 1923096 | 0.02 | 0.98 | 1668264 | 0.03 | 0.99 | 2846588 | 0.02 | 0.98 |
| 03P23A | 1912982 | 0.08 | 0.99 | 1443605 | 0.14 | 0.99 | 1380148 | 0.46 | 1.00 |
| 04F19A | 1648058 | 0.20 | 0.98 | 1397319 | 0.23 | 0.99 | 2990725 | 0.85 | 1.00 |
| 04H19B | 1748476 | 0.03 | 0.98 | 1485488 | 0.03 | 0.98 | 2081046 | 0.11 | 0.98 |
| 04N24A | 2329145 | 0.08 | 1.00 | 1648055 | 0.06 | 1.00 | 3533277 | 0.09 | 0.98 |
| 05F22A | 1635742 | 0.05 | 0.99 | 1157780 | 0.06 | 0.99 | 1307882 | 0.46 | 1.00 |
| 05K18A | 2276068 | 0.04 | 0.99 | 1686064 | 0.04 | 0.99 | 3294688 | 0.10 | 0.99 |
| 05L17A | 2605153 | 0.13 | 1.00 | 1820011 | 0.13 | 0.99 | 20643 | 0.00 | 0.39 |
| 05M23A | 1656376 | 0.02 | 0.97 | 1330589 | 0.02 | 0.98 | 2166761 | 0.08 | 0.96 |
| 06M22A | 1961592 | 0.28 | 0.99 | 1648736 | 0.23 | 1.00 | 3247802 | 0.14 | 0.97 |
| 06O22A | 2102518 | 0.04 | 1.00 | 1509126 | 0.02 | 0.98 | 3603647 | 0.08 | 0.98 |
| 07A08A | 2127682 | 0.06 | 1.00 | 1515404 | 0.04 | 0.99 | 2868973 | 0.08 | 0.98 |
| 07E07A | 2123998 | 0.11 | 0.99 | 1464418 | 0.08 | 0.99 | 3204993 | 0.18 | 1.00 |
| 08C23A | 1846944 | 0.16 | 0.99 | 1560224 | 0.15 | 1.00 | 2993023 | 0.22 | 1.00 |
| 08D04A | 1793421 | 0.08 | 0.99 | 1358475 | 0.08 | 0.99 | 1466485 | 0.22 | 0.98 |
| 09A16A | 2437151 | 0.12 | 1.00 | 1969113 | 0.13 | 0.99 | 2493167 | 0.09 | 0.98 |
| 09F14A | 2415648 | 0.11 | 0.98 | 1974105 | 0.11 | 0.99 | 3158896 | 0.13 | 0.98 |
| 09G09A | 1754110 | 0.08 | 0.98 | 1274166 | 0.06 | 0.95 | 2207821 | 0.07 | 1.00 |
| 10M10A | 2131362 | 0.31 | 0.99 | 1554524 | 0.29 | 1.00 | 2280639 | 0.75 | 1.00 |
| 11I06A | 1965753 | 0.02 | 0.97 | 1317659 | 0.02 | 0.98 | 32225 | 9.41 | 1.00 |
| 12P23A | 2152104 | 0.33 | 1.00 | 1501774 | 0.37 | 1.00 | 2274507 | 0.07 | 0.99 |
| rfmAb-1 | 1620036 | 0.07 | 0.98 | 1276399 | 0.07 | 0.99 | 2494335 | 0.06 | 0.96 |

As detailed in Table 25, twenty (20) out of twenty-two (22) mAbs showed high affinity for human, cyno monkey, and mouse MuSK. 05L17A and 11106A showed high affinity with human/cyno monkey MuSK, but low affinity with mouse MuSK.

Finally, the same twenty-two (22) mAbs were subjected to ELISA analysis to examine their binding sites on MuSK. High-binding 96-well plates (Corning, 9018) were coated with 100 μL per well of huMuSK ECD, huMuSK Fz, or huMuSK Ig3-Fz (2 μg/mL in PBS) overnight at 4° C. After 3 washes with 1× Phosphate-Buffered Saline, 0.1% Tween 20 (PBST), each plate was blocked with 1% BSA in PBS for 1 h at room temperature (RT). Diluents of anti-MuSK antibodies (3-fold serial dilutions with concentrations in the range of 0-100 nM) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. 3,3′, 5,5′-tetramethylbenzidine (TMB) Stabilized Chromogen substrate solution (Thermo Fisher Scientific, SB02) was added to the plate, and the reaction was stopped by adding 1M sulfuric acid. The absorbance at 450 nm was measured in a plate reader. The EC50 of each antibody against Fz, Ig3-Fz, and ECD of human MuSK was listed in Table 26 along with the predicted binding region.

TABLE 26
EC50 of anti-MuSK antibodies against Fz,
Ig3-Fz, and ECD of human MuSK in ELISA

| | Fz EC50 | Ig3-Fz EC50 | ECD EC50 | Predicted binding |
|---|---|---|---|---|
| ID | (nM) | (nM) | (nM) | region |
| 02E22A | 10.160 | 0.037 | 0.036 | Ig3 |
| 03F07A | 0.038 | 0.034 | 0.034 | Fz |
| 03P23A | 0.049 | 0.037 | 0.036 | Fz |
| 04F19A | 0.065 | 0.029 | 0.023 | Fz |
| 04H19B | 0.075 | 0.061 | 0.054 | Fz |
| 04N24A | 0.109 | 0.048 | 0.048 | Fz |
| 05F22A | 0.028 | 0.027 | 0.026 | Fz |
| 05K18A | 0.079 | 0.041 | 0.040 | Fz |
| 05L17A | 0.064 | 0.056 | 0.045 | Fz |
| 05M23A | 0.039 | 0.053 | 0.054 | Fz |
| 06M22A | 0.116 | 0.038 | 0.033 | Fz |
| 06O22A | 0.062 | 0.028 | 0.025 | Fz |
| 07A08A | 0.093 | 0.063 | 0.056 | Fz |
| 07E07A | 0.281 | 0.051 | 0.055 | Fz |
| 08C23A | 0.182 | 0.039 | 0.034 | Fz |
| 08D04A | UND | 0.025 | 0.023 | Ig3 |
| 09A16A | 0.134 | 0.053 | 0.047 | Fz |
| 09F14A | 0.075 | 0.035 | 0.034 | Fz |
| 09G09A | 0.169 | 0.031 | 0.031 | Fz |
| 10M10A | 0.078 | 0.033 | 0.033 | Fz |
| 11I06A | 0.042 | 0.037 | 0.033 | Fz |
| 12P23A | 0.073 | 0.044 | 0.045 | Fz |
| rfmAb-1 | 0.104 | 0.053 | 0.044 | Fz |

The present inventors estimated that antibodies with low affinity with Fz, but high affinity with Ig3-Fz and ECD likely bound to the Ig3 domain (e.g., 02E22A and 08D04A); while antibodies that had high affinity with Fz, Ig3-Fz, and ECD bound to the Fz domain of MuSK.

Eleven anti-MuSK agonist antibodies (02E22A, 03P23A, 04F19A, 04H19B, 04N24A, 07A08A, 08C23A, 09A16A, 09F14A, 09G09A, and 12P23A) were selected for further characterization to determine MuSK activation in muscle cells by assessing tyrosine phosphorylation of MuSK and induces clustering of Acetylcholine Receptor (AChR) at the neuromuscular junction in C2C12 myotubes.

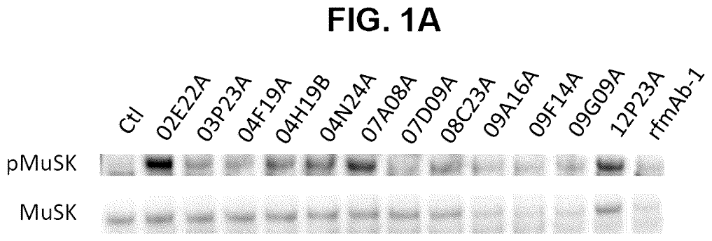

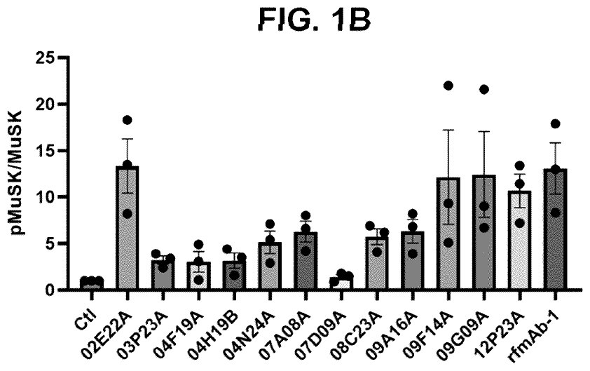

MuSK phosphorylation/activation. First, C2C12 mouse myoblast cells (ATCC, CRL-1772) were differentiated into myotubes. 2.5×10⁵ C2C12 cells were cultured in a 60 mm petri dish for 3 days in DMEM (Gibco, 11995-040) with 10% FBS. The cells were then differentiated by switching to differentiation medium containing DMEM (without sodium pyruvate) (Gibco, 31053-028) with 2% horse serum (Gibco, 26050-088) and incubation for 2 days. Next, differentiated C2C12 was treated with 10 nM of the anti-MuSK antibody for 30 min. After the treatment, cells were immediately lysed in 500 μL per dish of Pierce™ IP Lysis Buffer (Thermo Scientific, 87787) plus Halt™ Protease and Phosphatase Inhibitor Cocktail (Thermo Scientific, 78444). Cell lysate was centrifuged at 150,000 g for 5 min at 4° C. to remove any pellet. MuSK was immunoprecipitated from cell lysate with the human version of rfmAb-1 (hu-rfmAb-1). The immunoprecipitation was conducted using Dynabeads™ Protein G Immunoprecipitation Kit (Invirogen, 10007D) following manufacture's instruction. 5 μg hu-rfmAb-1 attached to 50 μL dynabeads was added to 500 μL cell lysate. Finally, immunoprecipitated samples were processed for Western Blot (WB). Samples were loaded into NuPAGE™ Bis-Tris 4-12% Mini Protein Gels (Invitrogen, NP0323BOX) and run at 200V for 35 min. Proteins were transferred to nitrocellulose membrane (Invitrogen, IB23001). The membrane was blocked with 1× TRIS-buffered saline (TBST) with 5% BSA for 1 h at RT and probed with an anti-phosphotyrosine antibody (EMD Millipore, 05-321) diluted 1:1000 in TBST with 5% BSA or with an anti-MuSK polyclonal antibody (R&D Systems, AF562) diluted 1:1000 in TBST with 5% BSA overnight at 4° C. After 3 washes in TBST, the membrane was incubated with goat anti-mouse IgG, HRP (Invitrogen, G21040) or donkey anti-goat IgG, HRP (Invitrogen, A15999) for 2 h at RT. After 3 washes in TBST, the protein bands on membrane were revealed with SuperSignal™ West Dura Extended Duration Substrate (Thermo Scientific, 34076). The membrane was imaged under Western Blot Imaging System Azure 600 (Azure Biosystems, AZI600-01). The result of the Western Blot is shown in FIG. 1A. Eleven (11) anti-MuSK agonist antibodies and reference antibody rfmAb-1 increased MuSK phosphorylation to various degrees with the greatest activation seen for 02E22A, 04H19B, 04N24A, 07A08A and 12P23A. 07D09A, a non-agonistic MuSK binder, had no effect on MuSK phosphorylation. The ratios of phospho-MuSK to total MuSK indicating levels of MuSK phosphorylation/activation were analyzed, which showed the greatest relative activation for 02E22A, 09F14A, 09G09A, 12P23A and rfmAb-1.

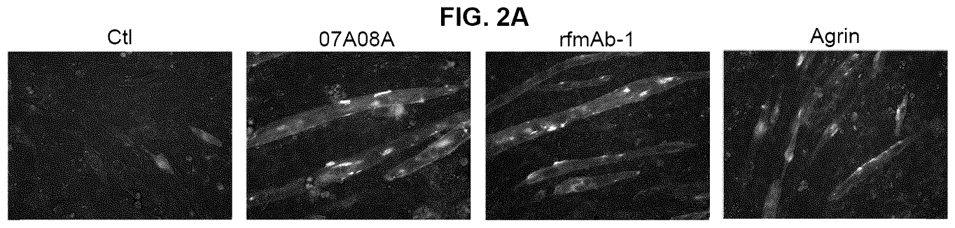

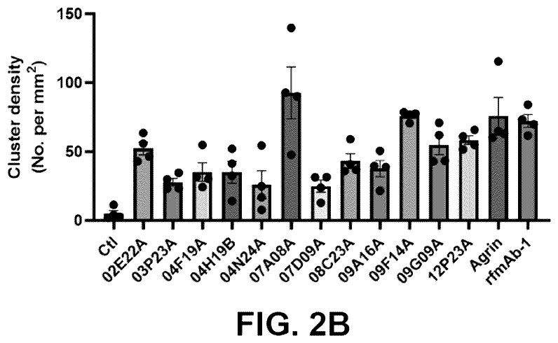

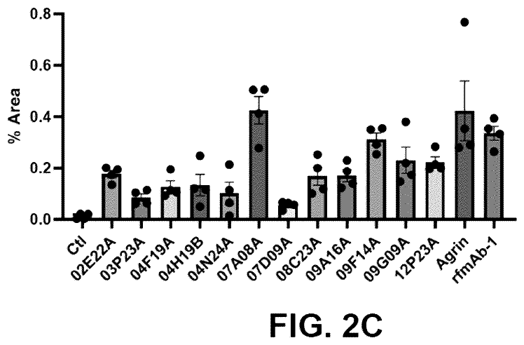

Acetylcholine Receptor (AChR) Clustering. MuSK activation induces clustering of Acetylcholine Receptor (AChR) at neuromuscular junction. To evaluate the effect of anti-MuSK antibody on AChR clustering, differentiated C2C12 myotubes were treated with an anti-MuSK antibody. AChR clustering on C2C12 was revealed by an AChR labelling dye. First, C2C12 cells were seeded in DMEM with 10% FBS in a chambered coverslip with 8 wells (Ibidi, 80806) at the seeding density of 1×10⁴ cells/cm². At 70% confluency, differentiation of C2C12 was induced by switching to differentiation medium containing DMEM (without sodium pyruvate) and 2% horse serum and incubation for 2 days. Differentiated C2C12 cells were treated with 1 nM of anti-MuSK antibody or rat Agrin (R&D systems, 550-AG-100) for 16 h. Cells were rinsed 2 times with differentiation medium, and incubated with 5 μg/mL of α-Bungarotoxin, Alexa Fluor™ 488 conjugate (Invitrogen, B13422) in differentiation medium for 30 min at 37° C. Cells were then washed with PBS, fixed with 4% Paraformaldehyde in PBS for 10 min, and washed again with PBS. Fixed cells were imaged under a wide-field fluorescence microscope. The AChR cluster density and percentage area of clusters in images were analyzed using ImageJ (version 1.53t). 8 randomly selected fields per well and 4 wells per treatment group were analyzed. The result of AChR clustering assay was shown in FIGS. 2A, 2B and 2C. Eleven anti-MuSK agonist antibodies, rfmAb-1, and Agrin increased AChR clustering to varying degrees with 07A08A showing the greatest increase of AChR clustering. 07D09A, a non-functional MuSK binder, had little effect on AChR clustering.

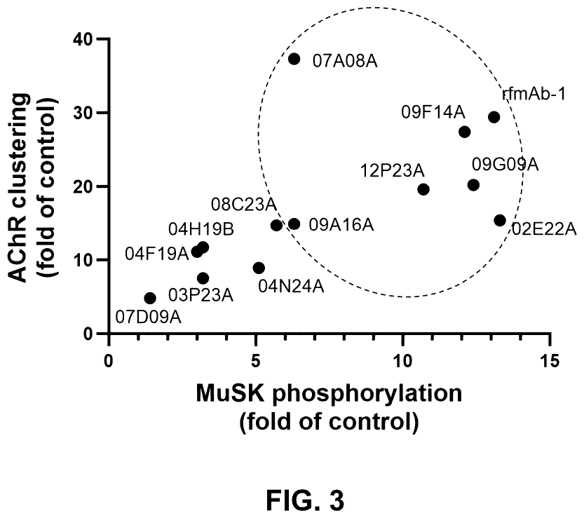

Correlation of MuSK Activation and AChR Clustering. To examine the correlation of MuSK activation and AChR clustering, the level of MuSK phosphorylation in C2C12 induced by an anti-MuSK antibody were plotted against the level of AChR clustering in C2C12 induced by the same antibody (FIG. 3). Interestingly, the antibodies with the greatest degree of MuSK activation did not necessarily result in the greatest degree of AChR clustering and vice versa. The antibodies at the top-right corner of the plot had the ability to induce strong MuSK activation and AChR clustering, while the antibodies at the bottom-left corner, such as the non-functional binder 07D09A, had little effect on MuSK activation or AChR clustering. Based on these findings, six inventive anti-MuSK agonistic antibodies were selected for further characterization.

Example 4. Pharmacokinetics of Anti-MuSK Agonistic Antibodies in Mice. PK assessment of six anti-MuSK agonistic antibodies showing strong MuSK activation and AChR clustering (02E22A, 07A08A, 09A16A, 09F14A, 09G09A, 12P23A) and one control (rfmAb-1) in C57BL/6J mice was completed. Antibodies were injected intraperitoneally (ip) in adult male C57BL/6J mice (The Jackson Laboratory, Strain #000664) at a dose of 10 mg/kg. Serum samples were collected at 0, 4, 8 hours, 1, 2, 3, 5, 7, 10, 14, 16, 18, and 21 days after the injection (Serum samples from 3 mice each antibody per time point). Antibody levels in serum were measured by ELISA with human MuSK ECD as the coated antigen. High-binding 96-well plates (Corning, 9018) were coated with 100 μL per well of 2 μg/mL huMuSK ECD overnight at 4° C. After 3 washes with PBST, each plate was blocked with 1% BSA in PBS for 1 h at RT. Serial dilutions of purified antibody (initial dilution 1.5 ng/ml, 3-fold dilutions, 8-points in duplicate) as standards or serum (initial dilution 1:500, 3-fold dilutions, 3-points) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. TMB Stabilized Chromogen substrate solution (Thermo Fisher Scientific, SB02) was added to the plate, and the reaction was stopped by adding 1M sulfuric acid. The absorbance at 450 nm was measured in a plate reader. Non-compartmental PK analysis was conducted via winNonlin software (version 8.3). The PK profiles of antibodies were shown in Table 27.

TABLE 27
Pharmacokinetic profiles of anti-MuSK agonistic antibodies.

| Parameter | Unit | 02E22A | 07A08A | 07D09A | 09A16A |
|---|---|---|---|---|---|
| Lambda_z | 1/h | 0.0023 | 0.0016 | 0.0023 | 0.0031 |
| t½ | h | 296 | 428 | 296 | 226 |
| Tmax | h | 8 | 24 | 8 | 48 |
| Cmax | μg/ml | 373 | 135 | 134 | 160 |
| C0 | μg/ml | 312 | 57 | 111 | 120 |
| Clast_obs/Cmax | | 0.0091 | 0.0429 | 0.0250 | 0.0073 |
| AUC 0-t | μg/ml*h | 54083 | 55223 | 50183 | 56988 |
| AUC 0-inf_obs | μg/ml*h | 55529 | 58798 | 51611 | 57370 |
| AUC 0-t/0-inf_obs | | 0.97 | 0.94 | 0.97 | 0.99 |
| AUMC 0-inf_obs | μg/ml*h\hat{ }2 | 20244851 | 29873801 | 20898556 | 18734559 |
| MRT 0-inf_obs | h | 365 | 508 | 405 | 327 |
| Vz_obs | (mg)/(μg/ml) | 6.35 | 8.66 | 6.83 | 4.69 |
| Cl_obs | (mg)/(μg/ml)/h | 0.01 | 0.01 | 0.02 | 0.01 |
| Vss_obs | (mg)/(μg/ml) | 5.42 | 7.13 | 6.47 | 4.70 |

| Parameter | 09F14A | 09G09A | 12P23A | rfmAb-1 |
|---|---|---|---|---|
| Lambda_z | 0.0020 | 0.0016 | 0.0044 | 0.0060 |
| t½ | 348 | 424 | 156 | 116 |
| Tmax | 4 | 4 | 4 | 48 |
| Cmax | 158 | 225 | 208 | 147 |
| C0 | 198 | 283 | 377 | 118 |
| Clast_obs/Cmax | 0.0370 | 0.0338 | 0.0030 | 0.0014 |
| AUC 0-t | 54238 | 73464 | 36431 | 31228 |
| AUC 0-inf_obs | 57164 | 78119 | 36574 | 31261 |
| AUC 0-t/0-inf_obs | 0.95 | 0.94 | 1.00 | 1.00 |
| AUMC 0-inf_obs | 27996130 | 40331194 | 7816689 | 5182537 |
| MRT 0-inf_obs | 490 | 516 | 214 | 166 |
| Vz_obs | 7.24 | 6.46 | 5.08 | 4.42 |
| Cl_obs | 0.01 | 0.01 | 0.02 | 0.03 |
| Vss_obs | 7.07 | 5.45 | 4.82 | 4.38 |

The half-life of 02E22A, 07A08A, 09A16A, 09F14A, 09G09A, and 12P23A was 296, 428, 296, 226, 348, 424, and 156 hours, respectively, all of which were significantly longer than the half-life of rfmAb-1 (116 hours). The Area Under the Curve (AUC) of these antibodies was also higher than that of rfmAb-1. These results suggest that 02E22A, 07A08A, 09A16A, 09F14A, 09G09A, and 12P23A exhibited favorable PK profiles in mice.

Example 5. Target engagement study of anti-MuSK agonistic antibodies. Anti-MuSK agonistic antibodies 02E22A, 07A08A, 09F14A, and 09G09A were selected for further characterization based on in-vitro functional and in-vivo pharmacokinetic profiles. The selected antibodies were converted into fully human IgG1 incorporating L234A, L235A, and P329G (LALAPG) mutations in the Fc region to abrogate effector function.

To evaluate whether the selected anti-MuSK agonistic antibodies target NMJ in-vivo, C57BL/6J mice at postnatal day 4 were injected intraperitoneally with 0.1, 1, or 10 mg/kg of human 02E22A, 07A08A, 09F14A, and 09G09A. 3 days after the injection, mice were euthanized, and diaphragm muscle was collected for immunohistostaining with Alpha-bungarotoxin to label acetylcholine receptor, a marker of NMJ, and with anti-human IgG to label the injected human antibody. The diaphragm muscle was fixed with 4% paraformaldehyde in a 12-well plate for 1h 40 min at room temperature (RT). After 3 washes with PBS, the muscle was washed with 0.1 M glycine for 1 h on a shaker (100 rpm) at RT to reduce the autofluorescence. After 3 washes with PBS, the diaphragm was permeabilized with 0.5% Triton X-100 in PBS for 1 h at room temperature without shaking. The diaphragm was then blocked with PBS containing 5% BSA and 0.5% Triton X-100 in a 24-well plate overnight at 4° C. After blocking, the diaphragm muscle was incubated with Alexa Fluor™ 594 Alpha-bungarotoxin (B13423, Invitrogen™, 1:1000 diluted in blocking buffer) and Alexa Fluor™ 488 Goat anti-Human IgG (A-11013, Invitrogen™, 1:500) in the 24-well plate overnight on a shaker (100 rpm) at 4° C. After 3 washes with 0.5% Triton X-100, the diaphragm muscle was mounted on a slide with 100 μL of 50% glycerol and covered with a coverslip. The slide was stored at 4° C. for confocal imaging. Unified parameters were used for confocal imaging. One 10× Z-stack and four 20× Z-stack images with different fields of view were collected on each sample. 16 NMJ structures/structural groups in each 20× field of view were randomly selected. The fluorescence intensity of alpha-bungarotoxin and human IgG in a single field of view was analyzed. The ratio of human IgG fluorescence intensity to alpha-bungarotoxin was calculated. The values of four fields of view in each sample were averaged.

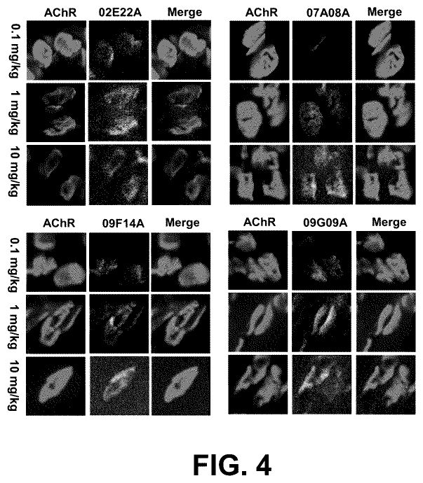

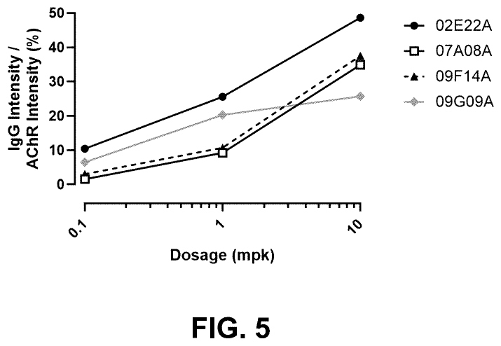

IHC Images of individual NMJs on diaphragm muscle collected 3 days after injection were shown in FIG. 4. The signals of all 4 human anti-MuSK antibodies overlapped well with the NMJ marker. The ratios of human IgG fluorescence intensity to AChR intensity on NMJ were analyzed (FIG. 5). The IgG signal on NMJ increased in a dose-dependent manner, which suggests a dose-dependent targeting of anti-MuSK antibodies to NMJ in-vivo.

### Embodiments

The present disclosure notes that various embodiments are disclosed herein, including:
A. An anti-MuSK antibody comprising:
a variable heavy chain comprising:
   a complementarity determining region (CDR) 1 with the amino acid sequence set forth in SEQ ID NOs: 3, 11, 19, 27, 35, 43, 51, 59, 67, 75, 83, 91, 99, 107, 115, 123, 131, 139, 147, 155, 163, or 171;
   a CDR2 with the amino acid sequence set forth in SEQ ID NOs: 4, 12, 20, 28, 36, 44, 52, 60, 68, 76, 84, 92, 100, 108, 116, 124, 132, 140, 148, 156, 164, or 172; and
   a CDR3 with the amino acid sequence set forth in SEQ ID NOs: 5, 13, 21, 29, 37, 45, 53, 61, 69, 77, 85, 93, 101, 109, 117, 125, 133, 141, 149, 157, 165, or 173; and
a variable light chain comprising:
   a CDR1 with the amino acid sequence set forth in SEQ ID NOs: 7, 15, 23, 31, 39, 47, 55, 63, 71, 79, 87, 95, 103, 111, 119, 127, 135, 143, 151, 159, 167, or 175;
   a CDR2 with the amino acid sequence set forth in SEQ ID NOs: 8, 16, 24, 32, 40, 48, 56, 64, 72, 80, 88, 96, 104, 112, 120, 128, 136, 144, 152, 160, 168, or 176; and
   a CDR3 with the amino acid sequence set forth in SEQ ID NOs: 9, 17, 25, 33, 41, 49, 57, 65, 73, 81, 89, 97, 105, 113, 121, 129, 137, 145, 153, 161, 169, or 177.
A1. The anti-MuSK antibody of embodiment A, wherein the variable heavy chain comprises a CDR1 with the amino acid sequence set forth in SEQ ID NO:3, a CDR2 with the amino acid sequence set forth in SEQ ID NO:4, and a CDR3 with the amino acid sequence set forth in SEQ ID NO:5; and the variable light chain comprises a CDR1 with the amino acid sequence set forth in SEQ ID NO:7, a CDR2 with the amino acid sequence set forth in SEQ ID NO:8, and a CDR3 with the amino acid sequence set forth in SEQ ID NO:9.
   A1 (i). The anti-MuSK antibody of embodiment A1, wherein one conservative substitution is present in SEQ ID NO: 3, 4, 5, 7, 8, and/or 9.
   A1 (ii). The anti-MuSK antibody of A1 or A1 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2
   A1 (iii). The anti-MuSK antibody of embodiment A1 or A1 (i), wherein the variable heavy chain comprises or consists of the amino acid sequence set forth in SEQ ID NO: 2.
   A1 (iv). The anti-MuSK antibody of any preceding embodiment of A1 (i.e., A1, A1 (i), A1 (ii), or A1 (iii)), wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.
   A1 (v). The anti-MuSK antibody of embodiment preceding embodiment of A1 (i.e., A1, A1 (i), A1 (ii), A1 (iii), or A1 (iv)), wherein the variable light chain comprises or consists of the amino acid sequence set forth in SEQ ID NO:6.
A2. The anti-MuSK antibody of embodiment A, wherein the variable heavy chain comprises a CDR1 with the amino acid sequence set forth in SEQ ID NO:11, a CDR2 with the amino acid sequence set forth in SEQ ID NO: 12, and a CDR3 with the amino acid sequence set forth in SEQ ID NO: 13; and the variable light chain comprises a CDR1 with the amino acid sequence set forth in SEQ ID NO:15, a CDR2 with the amino acid sequence set forth in SEQ ID NO: 16, and a CDR3 with the amino acid sequence set forth in SEQ ID NO: 17.
   A2 (i). The anti-MuSK antibody of embodiment A2, wherein one conservative substitution is present in SEQ ID NO: 11, 12, 13, 15, 16, and/or 17.
   A2 (ii). The anti-MuSK antibody of embodiment A2 or A2 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 10.
   A2 (iii). The anti-MuSK antibody of embodiment A2 or A2 (i), wherein the variable heavy chain comprises or consists of the amino acid sequence set forth in SEQ ID NO: 10.
   A2 (iv). The anti-MuSK antibody of any preceding embodiment of A2, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:14.
   A2 (v). The anti-MuSK antibody of any preceding embodiment of A2, wherein the variable light chain comprises or consists of the amino acid sequence set forth in SEQ ID NO: 14.
A3. The anti-MuSK antibody of embodiment A, wherein the variable heavy chain comprises a CDR1 with the amino acid sequence set forth in SEQ ID NO: 19, a CDR2 with the amino acid sequence set forth in SEQ ID NO:20, and a CDR3 with the amino acid sequence set forth in SEQ ID NO:21; and the variable light chain comprises a CDR1 with the amino acid sequence set forth in SEQ ID NO:23, a CDR2 with the amino acid sequence set forth in SEQ ID NO:24, and a CDR3 with the amino acid sequence set forth in SEQ ID NO:25.
   A3 (i). The anti-MuSK antibody of embodiment A3, wherein one conservative substitution is present in SEQ ID NO: 19, 20, 21, 23, 24, and/or 25.
   A3 (ii). The anti-MuSK antibody of embodiment A3 or A3 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18
   A3 (iii). The anti-MuSK antibody of embodiment A3 or A3 (i), wherein the variable heavy chain comprises or consists of the amino acid sequence set forth in SEQ ID NO: 18.
   A3 (iv). The anti-MuSK antibody of any preceding embodiment of A3, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:22.
   A3 (v). The anti-MuSK antibody of embodiment A3, wherein the variable light chain comprises or consists of the amino acid sequence set forth in SEQ ID NO:22.
A4. The anti-MuSK antibody of embodiment A, wherein the variable heavy chain comprises a CDR1 with the amino acid sequence set forth in SEQ ID NO:27, a CDR2 with the amino acid sequence set forth in SEQ ID NO:28, and a CDR3 with the amino acid sequence set forth in SEQ ID NO:29; and the variable light chain comprises a CDR1 with the amino acid sequence set forth in SEQ ID NO:31, a CDR2 with the amino acid sequence set forth in SEQ ID NO:32, and a CDR3 with the amino acid sequence set forth in SEQ ID NO:33.
   A4 (i). The anti-MuSK antibody of embodiment A4, wherein one conservative substitution is present in SEQ ID NO: 27, 28, 29, 31, 32, and/or 33.
   A4 (ii). The anti-MuSK antibody of embodiment A4 or A4 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:26
   A4 (iii). The anti-MuSK antibody of embodiment A4 or A4 (i), wherein the variable heavy chain comprises or consists of the amino acid sequence set forth in SEQ ID NO: 26.
   A4 (iv). The anti-MuSK antibody of any preceding embodiment of A4, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:30.
   A4 (v). The anti-MuSK antibody of any preceding embodiment of A4, wherein the variable light chain comprises or consists of the amino acid sequence set forth in SEQ ID NO:30.
A5. The anti-MuSK antibody of embodiment A, wherein the variable heavy chain comprises a CDR1 with the amino acid sequence set forth in SEQ ID NO:35, a CDR2 with the amino acid sequence set forth in SEQ ID NO:36, and a CDR3 with the amino acid sequence set forth in SEQ ID NO:37; and the variable light chain comprises a CDR1 with the amino acid sequence set forth in SEQ ID NO:39, a CDR2 with the amino acid sequence set forth in SEQ ID NO:40, and a CDR3 with the amino acid sequence set forth in SEQ ID NO:41.
   A5 (i). The anti-MuSK antibody of embodiment A5, wherein one conservative substitution is present in SEQ ID NO: 35, 36, 37, 39, 40, and/or 41.
   A5 (ii). The anti-MuSK antibody of embodiment A5 or A5 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:34
   A5 (iii). The anti-MuSK antibody of embodiment A5 or A5 (i), wherein the variable heavy chain comprises or consists of the amino acid sequence set forth in SEQ ID NO: 34.
   A5 (iv). The anti-MuSK antibody of any preceding embodiment of A5, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:38.
   A5 (v). The anti-MuSK antibody of preceding embodiment of A5, wherein the variable light chain comprises or consists of the amino acid sequence set forth in SEQ ID NO:38.
A6. The anti-MuSK antibody of embodiment A, wherein the variable heavy chain comprises a CDR1 with the amino acid sequence set forth in SEQ ID NO:43, a CDR2 with the amino acid sequence set forth in SEQ ID NO:44, and a CDR3 with the amino acid sequence set forth in SEQ ID NO:45; and the variable light chain comprises a CDR1 with the amino acid sequence set forth in SEQ ID NO:47, a CDR2 with the amino acid sequence set forth in SEQ ID NO:48, and a CDR3 with the amino acid sequence set forth in SEQ ID NO:49.
   A6 (i). The anti-MuSK antibody of embodiment A6, wherein one conservative substitution is present in SEQ ID NO: 43, 44, 45, 47, 48, and/or 49.
   A6 (ii). The anti-MuSK antibody of embodiment A6 or A6 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:42
   A6 (iii). The anti-MuSK antibody of embodiment A6 or A6 (i), wherein the variable heavy chain comprises or consists of the amino acid sequence set forth in SEQ ID NO: 42.
   A6 (iv). The anti-MuSK antibody of any preceding embodiment of A6, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:46.
   A6 (v). The anti-MuSK antibody of any preceding embodiment of A6, wherein the variable light chain comprises or consists of the amino acid sequence set forth in SEQ ID NO:46.
A7. The anti-MuSK antibody of embodiment A, wherein the variable heavy chain comprises a CDR1 with the amino acid sequence set forth in SEQ ID NO:51, a CDR2 with the amino acid sequence set forth in SEQ ID NO:52, and a CDR3 with the amino acid sequence set forth in SEQ ID NO:53; and the variable light chain comprises a CDR1 with the amino acid sequence set forth in SEQ ID NO:55, a CDR2 with the amino acid sequence set forth in SEQ ID NO:56, and a CDR3 with the amino acid sequence set forth in SEQ ID NO:57.
   A7 (i). The anti-MuSK antibody of embodiment A7, wherein one conservative substitution is present in SEQ ID NO: 51, 52, 53, 55, 56, and/or 57.
   A7 (ii). The anti-MuSK antibody of embodiment A7 or A7 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:50
   A7 (iii). The anti-MuSK antibody of embodiment A7 or A7 (i), wherein the variable heavy chain comprises or consists of the amino acid sequence set forth in SEQ ID NO: 50.
   A7 (iv). The anti-MuSK antibody of any preceding embodiment of A7, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:54.
   A7 (v). The anti-MuSK antibody of any preceding embodiment of A7, wherein the variable light chain comprises or consists of the amino acid sequence set forth in SEQ ID NO:54.
A8. The anti-MuSK antibody of embodiment A, wherein the variable heavy chain comprises a CDR1 with the amino acid sequence set forth in SEQ ID NO:59, a CDR2 with the amino acid sequence set forth in SEQ ID NO:60, and a CDR3 with the amino acid sequence set forth in SEQ ID NO:61; and the variable light chain comprises a CDR1 with the amino acid sequence set forth in SEQ ID NO:63, a CDR2 with the amino acid sequence set forth in SEQ ID NO:64, and a CDR3 with the amino acid sequence set forth in SEQ ID NO:65.
   A8 (i). The anti-MuSK antibody of embodiment A8, wherein one conservative substitution is present in SEQ ID NO: 59, 60, 61, 63, 64, and/or 65.
   A8 (ii). The anti-MuSK antibody of embodiment A8 or A8 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:58
   A8 (iii). The anti-MuSK antibody of embodiment A8 or A8 (i), wherein the variable heavy chain comprises or consists of the amino acid sequence set forth in SEQ ID NO: 58.
   A8 (iv). The anti-MuSK antibody of any preceding embodiment of A8, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:62.
   A8 (v). The anti-MuSK antibody of any preceding embodiment of A8, wherein the variable light chain comprises or consists of the amino acid sequence set forth in SEQ ID NO:62.
A9. The anti-MuSK antibody of embodiment A, wherein the variable heavy chain comprises a CDR1 with the amino acid sequence set forth in SEQ ID NO:67, a CDR2 with the amino acid sequence set forth in SEQ ID NO:68, and a CDR3 with the amino acid sequence set forth in SEQ ID NO:69; and the variable light chain comprises a CDR1 with the amino acid sequence set forth in SEQ ID NO:71, a CDR2 with the amino acid sequence set forth in SEQ ID NO:72, and a CDR3 with the amino acid sequence set forth in SEQ ID NO:73.
   A9 (i). The anti-MuSK antibody of embodiment A9, wherein one conservative substitution is present in SEQ ID NO: 67, 68, 69, 71, 72, and/or 73.
   A9 (ii). The anti-MuSK antibody of embodiment A9 or A9 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:66
   A9 (iii). The anti-MuSK antibody of embodiment A9 or A9 (i), wherein the variable heavy chain comprises or consists of the amino acid sequence set forth in SEQ ID NO: 66.
   A9 (iv). The anti-MuSK antibody of any preceding embodiment of A9, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:70.
   A9 (v). The anti-MuSK antibody of any preceding embodiment of A9, wherein the variable light chain comprises or consists of the amino acid sequence set forth in SEQ ID NO:70.
A10. The anti-MuSK antibody of embodiment A, wherein the variable heavy chain comprises a CDR1 with the amino acid sequence set forth in SEQ ID NO:75, a CDR2 with the amino acid sequence set forth in SEQ ID NO:76, and a CDR3 with the amino acid sequence set forth in SEQ ID NO:77; and the variable light chain comprises a CDR1 with the amino acid sequence set forth in SEQ ID NO:79, a CDR2 with the amino acid sequence set forth in SEQ ID NO:80, and a CDR3 with the amino acid sequence set forth in SEQ ID NO:81.
   A10 (i). The anti-MuSK antibody of embodiment A10, wherein one conservative substitution is present in SEQ ID NO: 75, 76, 77, 79, 80, and/or 81.
   A10 (ii). The anti-MuSK antibody of embodiment A10 or A10 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:74
   A10 (iii). The anti-MuSK antibody of embodiment A10 or A10 (i), wherein the variable heavy chain comprises or consists of the amino acid sequence set forth in SEQ ID NO:74.
   A10 (iv). The anti-MuSK antibody of any preceding embodiment of A10, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:78
   A10 (v). The anti-MuSK antibody of any preceding embodiment of
A10, wherein the variable light chain comprises or consists of the amino acid sequence set forth in SEQ ID NO:78.
A11. The anti-MuSK antibody of embodiment A, wherein the variable heavy chain comprises a CDR1 with the amino acid sequence set forth in SEQ ID NO:83, a CDR2 with the amino acid sequence set forth in SEQ ID NO:84, and a CDR3 with the amino acid sequence set forth in SEQ ID NO:85; and the variable light chain comprises a CDR1 with the amino acid sequence set forth in SEQ ID NO:87, a CDR2 with the amino acid sequence set forth in SEQ ID NO:88, and a CDR3 with the amino acid sequence set forth in SEQ ID NO:89.
   A11 (i). The anti-MuSK antibody of embodiment A11, wherein one conservative substitution is present in SEQ ID NO: 83, 84, 85, 87, 88, and/or 89.
   A11 (ii). The anti-MuSK antibody of embodiment A11 or A11 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:82.
   A11 (iii). The anti-MuSK antibody of embodiment A11 or A11 (i), wherein the variable heavy chain comprises or consists of the amino acid sequence set forth in SEQ ID NO:82.
   A11 (iv). The anti-MuSK antibody of any preceding embodiment of A11, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:86.
   A11 (v). The anti-MuSK antibody of any preceding embodiment of A11, wherein the variable light chain comprises or consists of the amino acid sequence set forth in SEQ ID NO:86.
A12. The anti-MuSK antibody of embodiment A, wherein the variable heavy chain comprises a CDR1 with the amino acid sequence set forth in SEQ ID NO:91, a CDR2 with the amino acid sequence set forth in SEQ ID NO:92, and a CDR3 with the amino acid sequence set forth in SEQ ID NO:93; and the variable light chain comprises a CDR1 with the amino acid sequence set forth in SEQ ID NO:95, a CDR2 with the amino acid sequence set forth in SEQ ID NO:96, and a CDR3 with the amino acid sequence set forth in SEQ ID NO:97.
   A12 (i). The anti-MuSK antibody of embodiment A12, wherein one conservative substitution is present in SEQ ID NO: 91, 92, 93, 95, 96, and/or 97.
   A12 (ii). The anti-MuSK antibody of embodiment A12 or A12 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:90.
   A12 (iii). The anti-MuSK antibody of embodiment A12 or A12 (i), wherein the variable heavy chain comprises or consists of the amino acid sequence set forth in SEQ ID NO:90.
   A12 (iv). The anti-MuSK antibody of any preceding embodiment of A12, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:94.
   A12 (v). The anti-MuSK antibody of any preceding embodiment of A12, wherein the variable light chain comprises or consists of the amino acid sequence set forth in SEQ ID NO:94.
A13. The anti-MuSK antibody of embodiment A, wherein the variable heavy chain comprises a CDR1 with the amino acid sequence set forth in SEQ ID NO:99, a CDR2 with the amino acid sequence set forth in SEQ ID NO: 100, and a CDR3 with the amino acid sequence set forth in SEQ ID NO: 101; and the variable light chain comprises a CDR1 with the amino acid sequence set forth in SEQ ID NO: 103, a CDR2 with the amino acid sequence set forth in SEQ ID NO: 104, and a CDR3 with the amino acid sequence set forth in SEQ ID NO: 105.
   A13 (i). The anti-MuSK antibody of embodiment A13, wherein one conservative substitution is present in SEQ ID NO: 99, 100, 101, 103, 104, and/or 105.
   A13 (ii). The anti-MuSK antibody of embodiment A13 or A13 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:98.
   A13 (iii). The anti-MuSK antibody of embodiment A13 or A13 (i), wherein the variable heavy chain comprises or consists of the amino acid sequence set forth in SEQ ID NO:98.
   A13 (iv). The anti-MuSK antibody of any preceding embodiment of A13, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:102.
   A13 (v). The anti-MuSK antibody of any preceding embodiment of A13, wherein the variable light chain comprises or consists of the amino acid sequence set forth in SEQ ID NO: 102.
A14. The anti-MuSK antibody of embodiment A, wherein the variable heavy chain comprises a CDR1 with the amino acid sequence set forth in SEQ ID NO: 107, a CDR2 with the amino acid sequence set forth in SEQ ID NO: 108, and a CDR3 with the amino acid sequence set forth in SEQ ID NO: 109; and the variable light chain comprises a CDR1 with the amino acid sequence set forth in SEQ ID NO:111, a CDR2 with the amino acid sequence set forth in SEQ ID NO: 112, and a CDR3 with the amino acid sequence set forth in SEQ ID NO:113.
   A14 (i). The anti-MuSK antibody of embodiment A14, wherein one conservative substitution is present in SEQ ID NO: 107, 108, 109, 111, 112, and/or 113.
   A14 (ii). The anti-MuSK antibody of embodiment A14 or A14 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 106.
   A14 (iii). The anti-MuSK antibody of embodiment A14 or A14 (i), wherein the variable heavy chain comprises or consists of the amino acid sequence set forth in SEQ ID NO: 106.
   A14 (iv). The anti-MuSK antibody of any preceding embodiment of A14, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 110
   A14 (v). The anti-MuSK antibody of any preceding embodiment of A14, wherein the variable light chain comprises or consists of the amino acid sequence set forth in SEQ ID NO:110.
A15. The anti-MuSK antibody of embodiment A, wherein the variable heavy chain comprises a CDR1 with the amino acid sequence set forth in SEQ ID NO:115, a CDR2 with the amino acid sequence set forth in SEQ ID NO: 116, and a CDR3 with the amino acid sequence set forth in SEQ ID NO: 117; and the variable light chain comprises a CDR1 with the amino acid sequence set forth in SEQ ID NO: 119, a CDR2 with the amino acid sequence set forth in SEQ ID NO: 120, and a CDR3 with the amino acid sequence set forth in SEQ ID NO: 121.
   A15 (i). The anti-MuSK antibody of embodiment A15, wherein one conservative substitution is present in SEQ ID NO: 115, 116, 117, 119, 120, and/or 121.
   A15 (ii). The anti-MuSK antibody of embodiment A15 or A15 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 114.
   A15 (iii). The anti-MuSK antibody of embodiment A15 or A15 (i), wherein the variable heavy chain comprises or consists of the amino acid sequence set forth in SEQ ID NO: 114.
   A15 (iv). The anti-MuSK antibody of any preceding embodiment of A15, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 118.
   A15 (v). The anti-MuSK antibody of any preceding embodiment of A15, wherein the variable light chain comprises or consists of the amino acid sequence set forth in SEQ ID NO: 118.
A16. The anti-MuSK antibody of embodiment A, wherein the variable heavy chain comprises a CDR1 with the amino acid sequence set forth in SEQ ID NO: 123, a CDR2 with the amino acid sequence set forth in SEQ ID NO: 124, and a CDR3 with the amino acid sequence set forth in SEQ ID NO: 125; and the variable light chain comprises a CDR1 with the amino acid sequence set forth in SEQ ID NO: 127, a CDR2 with the amino acid sequence set forth in SEQ ID NO: 128, and a CDR3 with the amino acid sequence set forth in SEQ ID NO: 129.
   A16 (i). The anti-MuSK antibody of embodiment A16, wherein one conservative substitution is present in SEQ ID NO: 123, 124, 125, 127, 128, and/or 129.
   A16 (ii). The anti-MuSK antibody of embodiment A16 or A16 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 122.
   A16 (iii). The anti-MuSK antibody of embodiment A16 or A16 (i), wherein the variable heavy chain comprises or consists of the amino acid sequence set forth in SEQ ID NO: 122.
   A16 (iv). The anti-MuSK antibody of any preceding embodiment of A16, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:126.
   A16 (v). The anti-MuSK antibody of any preceding embodiment of A16, wherein the variable light chain comprises or consists of the amino acid sequence set forth in SEQ ID NO:126.
A17. The anti-MuSK antibody of embodiment A, wherein the variable heavy chain comprises a CDR1 with the amino acid sequence set forth in SEQ ID NO:131, a CDR2 with the amino acid sequence set forth in SEQ ID NO: 132, and a CDR3 with the amino acid sequence set forth in SEQ ID NO: 133; and the variable light chain comprises a CDR1 with the amino acid sequence set forth in SEQ ID NO: 135, a CDR2 with the amino acid sequence set forth in SEQ ID NO: 136, and a CDR3 with the amino acid sequence set forth in SEQ ID NO: 137.
   A17 (i). The anti-MuSK antibody of embodiment A17, wherein one conservative substitution is present in SEQ ID NO: 131, 132, 133, 135, 136, and/or 137.
   A17 (ii). The anti-MuSK antibody of embodiment A17 or A17 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:130.
   A17 (iii). The anti-MuSK antibody of embodiment A17 or A17 (i), wherein the variable heavy chain comprises or consists of the amino acid sequence set forth in SEQ ID NO: 130.
   A17 (iv). The anti-MuSK antibody of any preceding embodiment of A17, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 134.
   A17 (v). The anti-MuSK antibody of any preceding embodiment of A17, wherein the variable light chain comprises or consists of the amino acid sequence set forth in SEQ ID NO: 134.
A18. The anti-MuSK antibody of embodiment A, wherein the variable heavy chain comprises a CDR1 with the amino acid sequence set forth in SEQ ID NO: 139, a CDR2 with the amino acid sequence set forth in SEQ ID NO: 140, and a CDR3 with the amino acid sequence set forth in SEQ ID NO: 141; and the variable light chain comprises a CDR1 with the amino acid sequence set forth in SEQ ID NO: 143, a CDR2 with the amino acid sequence set forth in SEQ ID NO: 144, and a CDR3 with the amino acid sequence set forth in SEQ ID NO: 145.
   A18 (i). The anti-MuSK antibody of embodiment A18, wherein one conservative substitution is present in SEQ ID NO: 139, 140, 141, 143, 144, and/or 145.
   A18 (ii). The anti-MuSK antibody of embodiment A18 or A18 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 138.
   A18 (iii). The anti-MuSK antibody of embodiment A18 or A18 (i), wherein the variable heavy chain comprises or consists of the amino acid sequence set forth in SEQ ID NO: 138.
   A18 (iv). The anti-MuSK antibody of any preceding embodiment of A18, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 142.
   A18 (v). The anti-MuSK antibody of any preceding embodiment of A18, wherein the variable light chain comprises or consists of the amino acid sequence set forth in SEQ ID NO: 142.
A19. The anti-MuSK antibody of embodiment A, wherein the variable heavy chain comprises a CDR1 with the amino acid sequence set forth in SEQ ID NO:147, a CDR2 with the amino acid sequence set forth in SEQ ID NO: 148, and a CDR3 with the amino acid sequence set forth in SEQ ID NO: 149; and the variable light chain comprises a CDR1 with the amino acid sequence set forth in SEQ ID NO: 151, a CDR2 with the amino acid sequence set forth in SEQ ID NO: 152, and a CDR3 with the amino acid sequence set forth in SEQ ID NO: 153.
   A19 (i). The anti-MuSK antibody of embodiment A19, wherein one conservative substitution is present in SEQ ID NO: 147, 148, 149, 151, 152, and/or 153.
   A19 (ii). The anti-MuSK antibody of embodiment A19 or A19 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:146.
   A19 (iii). The anti-MuSK antibody of embodiment A19 or A19 (i), wherein the variable heavy chain comprises or consists of the amino acid sequence set forth in SEQ ID NO: 146.
   A19 (iv). The anti-MuSK antibody of any preceding embodiment of A19, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 150.
   A19 (v). The anti-MuSK antibody of any preceding embodiment of A19, wherein the variable light chain comprises or consists of the amino acid sequence set forth in SEQ ID NO: 150.
A20. The anti-MuSK antibody of embodiment A, wherein the variable heavy chain comprises a CDR1 with the amino acid sequence set forth in SEQ ID NO: 155, a CDR2 with the amino acid sequence set forth in SEQ ID NO: 156, and a CDR3 with the amino acid sequence set forth in SEQ ID NO: 157; and the variable light chain comprises a CDR1 with the amino acid sequence set forth in SEQ ID NO: 159, a CDR2 with the amino acid sequence set forth in SEQ ID NO: 160, and a CDR3 with the amino acid sequence set forth in SEQ ID NO: 161.
   A20 (i). The anti-MuSK antibody of embodiment A20, wherein one conservative substitution is present in SEQ ID NO: 155, 156, 157, 159, 160, and/or 161.
   A20 (ii). The anti-MuSK antibody of embodiment A20 or A20 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:154.
   A20 (iii). The anti-MuSK antibody of embodiment A20 or A20 (i), wherein the variable heavy chain comprises or consists of the amino acid sequence set forth in SEQ ID NO: 154.
   A20 (iv). The anti-MuSK antibody of any preceding embodiment of A20, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:158.
   A20 (v). The anti-MuSK antibody of any preceding embodiment of A20, wherein the variable light chain comprises or consists of the amino acid sequence set forth in SEQ ID NO: 158.
A21. The anti-MuSK antibody of embodiment A, wherein the variable heavy chain comprises a CDR1 with the amino acid sequence set forth in SEQ ID NO:163, a CDR2 with the amino acid sequence set forth in SEQ ID NO: 164, and a CDR3 with the amino acid sequence set forth in SEQ ID NO: 165; and the variable light chain comprises a CDR1 with the amino acid sequence set forth in SEQ ID NO: 167, a CDR2 with the amino acid sequence set forth in SEQ ID NO: 168, and a CDR3 with the amino acid sequence set forth in SEQ ID NO: 169.
   A21 (i). The anti-MuSK antibody of embodiment A21, wherein one conservative substitution is present in SEQ ID NO: 163, 164, 165, 167, 168, and/or 169.
   A21 (ii). The anti-MuSK antibody of embodiment A21 or A21 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 162.
   A21 (iii). The anti-MuSK antibody of embodiment A21 or A21 (i), wherein the variable heavy chain comprises or consists of the amino acid sequence set forth in SEQ ID NO: 162.
   A21 (iv). The anti-MuSK antibody of any preceding embodiment of A21, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:166.
   A21 (v). The anti-MuSK antibody of any preceding embodiment of A21, wherein the variable light chain comprises or consists of the amino acid sequence set forth in SEQ ID NO: 166.
A22. The anti-MuSK antibody of embodiment A, wherein the variable heavy chain comprises a CDR1 with the amino acid sequence set forth in SEQ ID NO:171, a CDR2 with the amino acid sequence set forth in SEQ ID NO: 172, and a CDR3 with the amino acid sequence set forth in SEQ ID NO: 173; and the variable light chain comprises a CDR1 with the amino acid sequence set forth in SEQ ID NO: 175, a CDR2 with the amino acid sequence set forth in SEQ ID NO: 176, and a CDR3 with the amino acid sequence set forth in SEQ ID NO: 177.
   A22 (i). The anti-MuSK antibody of embodiment A22, wherein one conservative substitution is present in SEQ ID NO: 171, 172, 173, 175, 176, and/or 177.
   A22 (ii). The anti-MuSK antibody of embodiment A22 or A22 (i), wherein the variable heavy chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:170.
   A22 (iii). The anti-MuSK antibody of embodiment A22 or A22 (i), wherein the variable heavy chain comprises or consists of the amino acid sequence set forth in SEQ ID NO: 170.
   A22 (iv). The anti-MuSK antibody of any preceding embodiment of A22, wherein the variable light chain comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:174.
   A22 (v). The anti-MuSK antibody of any preceding embodiment of A22, wherein the variable light chain comprises or consists of the amino acid sequence set forth in SEQ ID NO: 174.
B. The anti-MuSK antibody of any preceding embodiment, wherein the antibody is capable of binding MuSK (SEQ ID NO:1) or a variant thereof.
B1. The anti-MuSK antibody of embodiment B, wherein the antibody binds an epitope of the Ig-3 domain.
B2. The anti-MuSK antibody of embodiment B or B1, wherein the antibody binds an epitope of the FZ-CRD domain.
C. The anti-MuSK antibody of any preceding embodiment, wherein the antibody is capable of activating the signaling and/or phosphorylation of MuSK.
C1. The anti-MuSK antibody of embodiment C, wherein the phosphorylation comprises phosphorylation of β-subunits of MuSK.
C2. The anti-MuSK antibody of embodiment C1, wherein the phosphorylation of β-subunits of MuSK comprises phosphorylation of one or more tyrosine resides.
D. The anti-MuSK antibody of any preceding embodiment, wherein the antibody is a monoclonal antibody.
D1. The anti-MuSK antibody of embodiment D, wherein the monoclonal antibody is human or fully human.
D2. The anti-MuSK antibody of embodiment D, wherein the monoclonal antibody is humanized.
D3. The anti-MuSK antibody of any preceding embodiment, wherein the antibody comprises LALAPG mutation in the Fc region.
E. The anti-MuSK antibody of any preceding embodiment, wherein the antibody is an agonistic antibody.
E1. The anti-MusK antibody of embodiment E, wherein the antibody is useful to restore and/or increase MuSK function.
E2. The anti-MuSK antibody of embodiment E1, wherein MuSK function is increased or restored via enlargement of nerve terminals at NMJs.
E3. The anti-MuSK antibody of embodiment E1 or E2, wherein MuSK function is increased or restored via reversal of denervation at NMJs.
F. The anti-MuSK antibody of any preceding embodiment, wherein the antibody increases AChR clustering.
G. The anti-MuSK antibody of any preceding embodiment, wherein the antibody counteracts one or more natural autoantibodies that target and block the function of MuSK.
G1. The anti-MuSK antibody of embodiment G, wherein the one or more autoantibodies are IgG antibodies.
G2. The anti-MuSK antibody of embodiment G or G1, wherein the one or more autoantibodies are selected from the group consisting of IgG1, IgG2, IgG3, and IgG4.
G3. The anti-MuSK antibody of embodiment G, G1, or G2, wherein the one or more autoantibodies are of the IgG4 subclass.
G4. The anti-MuSK antibody of any preceding embodiment of G, wherein the one or more autoantibodies are against MuSK.
G5. The anti-MuSK antibody of any preceding embodiment of G, wherein the one or more autoantibodies are against LRP4.
G6. The anti-MuSK antibody of any preceding embodiment of G, wherein the one or more autoantibodies are against agrin.
H. A nucleic acid comprising a nucleotide sequence encoding an anti-MuSK antibody of any preceding embodiment.
I. A host cell transformed with a vector comprising a nucleic acid according to embodiment H.
I1. The host cell of embodiment I, wherein the host cell is a eukaryotic cell.
J. A method of producing an anti-MuSK antibody of embodiments A to G3, comprising culturing a host cell of embodiment I or I1 and recovering the anti-MuSK antibody.

What is claimed is:

1. An anti-MuSK antibody comprising:
a variable heavy (VH) chain comprising:
a complementarity determining region (CDR) 1 with the amino acid sequence set forth in SEQ ID NOs: 3, 11, 19, 27, 35, 43, 51, 59, 67, 75, 83, 91, 99, 107, 115, 123, 131, 139, 147, 155, 163, or 171;
a CDR2 with the amino acid sequence set forth in SEQ ID NOs: 4, 12, 20, 28, 36, 44, 52, 60, 68, 76, 84, 92, 100, 108, 116, 124, 132, 140, 148, 156, 164, or 172; and
a CDR3 with the amino acid sequence set forth in SEQ ID NOs: 5, 13, 21, 29, 37, 45, 53, 61, 69, 77, 85, 93, 101, 109, 117, 125, 133, 141, 149, 157, 165, or 173; and
a variable light (VL) chain comprising:
a CDR1 with the amino acid sequence set forth in SEQ ID NOs: 7, 15, 23, 31, 39, 47, 55, 63, 71, 79, 87, 95, 103, 111, 119, 127, 135, 143, 151, 159, 167, or 175;
a CDR2 with the amino acid sequence set forth in SEQ ID NOs: 8, 16, 24, 32, 40, 48, 56, 64, 72, 80, 88, 96, 104, 112, 120, 128, 136, 144, 152, 160, 168, or 176; and
a CDR3 with the amino acid sequence set forth in SEQ ID NOs: 9, 17, 25, 33, 41, 49, 57, 65, 73, 81, 89, 97, 105, 113, 121, 129, 137, 145, 153, 161, 169, or 177.

2. The anti-MuSK antibody of claim 1, wherein the VH chain comprises CDR1 with the amino acid sequence set forth in SEQ ID NO: 99, CDR2 with the amino acid sequence set forth in SEQ ID NO: 100, and CDR3 with the amino acid sequence set forth in SEQ ID NO: 101; and wherein the VL chain comprises CDR1 with the amino acid sequence set forth in SEQ ID NO: 103, CDR2 with the amino acid sequence set forth in SEQ ID NO: 104, and CDR3 with the amino acid sequence set forth in SEQ ID NO: 105.

3. The anti-MuSK antibody of claim 2, wherein the VH chain comprises the amino acid sequence set forth in SEQ ID NO: 98.

4. The anti-MuSK antibody of claim 2, wherein the VL chain comprises the amino acid sequence set forth in SEQ ID NO: 102.

5. The anti-MuSK antibody of claim 2, wherein the VH consists of the amino acid sequence of SEQ ID NO: 98 and the VL chain consists of the amino acid sequence of SEQ ID NO: 102.

6. The anti-MuSK antibody of claim 1, wherein the VH chain comprises CDR1 with the amino acid sequence set forth in SEQ ID NO: 139, CDR2 with the amino acid sequence set forth in SEQ ID NO: 140, and CDR3 with the amino acid sequence set forth in SEQ ID NO: 141; and wherein the VL chain comprises CDR1 with the amino acid sequence set forth in SEQ ID NO: 143, CDR2 with the amino acid sequence set forth in SEQ ID NO: 144, and CDR3 with the amino acid sequence set forth in SEQ ID NO: 145.

7. The anti-MuSK antibody of claim 6, wherein the VH chain comprises the amino acid sequence set forth in SEQ ID NO: 138.

8. The anti-MuSK antibody of claim 6, wherein the VL chain comprises the amino acid sequence set forth in SEQ ID NO: 142.

9. The anti-MuSK antibody of claim 6, wherein the VH chain consists of the amino acid sequence set forth in SEQ ID NO: 138 and the VL chain consists of the amino acid sequence set forth in SEQ ID NO: 142.

10. The anti-MuSK antibody of claim 1, wherein the VH chain comprises CDR1 with the amino acid sequence set forth in SEQ ID NO: 147, CDR2 with the amino acid sequence set forth in SEQ ID NO: 148, and CDR3 with the amino acid sequence set forth in SEQ ID NO: 149; and wherein the VL chain comprises CDR1 with the amino acid sequence set forth in SEQ ID NO: 151, CDR2 with the amino acid sequence set forth in SEQ ID NO: 152, and CDR3 with the amino acid sequence set forth in SEQ ID NO: 153.

11. The anti-MuSK antibody of claim 10, wherein the VH chain comprises the amino acid sequence set forth in SEQ ID NO: 146.

12. The anti-MuSK antibody of claim 10, wherein the VL chain comprises the amino acid sequence set forth in SEQ ID NO: 150.

13. The anti-MuSK antibody of claim 10, wherein the VH chain consists of the amino acid sequence set forth in SEQ ID NO: 146 and the VL chain consists of the amino acid sequence set forth in SEQ ID NO: 150.

14. The anti-MuSK antibody of claim 1, further comprising a LALAPG mutation in the Fc region.

15. The anti-MuSK antibody of claim 1, wherein the antibody is an agonistic antibody.

16. The anti-MuSK antibody of claim 1, wherein the antibody is a monoclonal antibody.

17. The anti-MuSK antibody of claim 16, wherein the monoclonal antibody is human or humanized.

18. A nucleic acid comprising a nucleotide sequence encoding an anti-MuSK antibody of claim 1.

19. A host cell transformed with a vector comprising a nucleic acid of claim 19.

20. A method of producing an anti-MuSK antibody, comprising culturing a host cell of claim 19 and recovering the anti-MuSK antibody.


SEQUENCE LISTING (177 sequences)

SEQ ID NO: 1 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 2 - protein, 115 aa, synthetic construct
EVQLVESGGGLVQPGGSLRLSCAASGFIFSIYWMSWVRQAPGKGLEWVANINQDGSEKYYVDSVKGRFTISRDNAKNSLYLQMNSLRAEDTAVYYCAREVGTTDWGQGTLVTVSS

SEQ ID NO: 3 - protein, 8 aa, synthetic construct
GFIFSIYW

SEQ ID NO: 4 - protein, 8 aa, synthetic construct
INQDGSEK

SEQ ID NO: 5 - protein, 8 aa, synthetic construct
AREVGTTD

SEQ ID NO: 6 - protein, 110 aa, synthetic construct
NFMLTQPHSVSESPGKTVTISCTRSSGGIASNYVQWYQQRPGSSPTTVIYEDNQRPSGVPDRFSGSIDSSSNSASLTISGLKTEDEADYYCQSYDSSNWVLGGGTKLTVL

SEQ ID NO: 7 - protein, 8 aa, synthetic construct
SGGIASNY

SEQ ID NO: 8 - skipped

SEQ ID NO: 9 - protein, 9 aa, synthetic construct
QSYDSSNWV

SEQ ID NO: 10 - protein, 120 aa, synthetic construct
QVQLQESGPGLVKPSETLSLTCTVSGGSISSYYWSWIRQPPGKGLEWIGYIYFSGRTYYNPSLKSRVTISVDTSKNQFSLKLSSVTAADTAVYYCARGGGLGISNWFDPWGQGTLVTVSS

SEQ ID NO: 11 - protein, 8 aa, synthetic construct
GGSISSYY

SEQ ID NO: 12 - protein, 7 aa, synthetic construct
IYFSGRT

SEQ ID NO: 13 - protein, 14 aa, synthetic construct
ARGGGLGISNWFDP

SEQ ID NO: 14 - protein, 108 aa, synthetic construct
SYELTQPPSVSVSPGQTARITCSGDALPKQYAYWYQQKPGQAPVLVIYKDSERPSGIPERFSGSSSGTTVTLTISGVQAEDEADYYCQSADSSGTYWVFGGGTKLTVL

SEQ ID NO: 15 - protein, 6 aa, synthetic construct
ALPKQY

SEQ ID NO: 16 - skipped

SEQ ID NO: 17 - protein, 11 aa, synthetic construct
QSADSSGTYWV

SEQ ID NO: 18 - protein, 124 aa, synthetic construct
QVQLVQSGAEVKKPGSSVKVSCKASGGTFSSYGISWVRQAPGQGLEWMGRIIPIFGTANYAQKFQGRVTITADKSTSTAYMELSSLRSEDTAVYYCARSRFLEWLLNYYGMDVWGQGTTVTVSS

SEQ ID NO: 19 - protein, 8 aa, synthetic construct
GGTFSSYG

SEQ ID NO: 20 - protein, 8 aa, synthetic construct
IIPIFGTA

SEQ ID NO: 21 - protein, 17 aa, synthetic construct
ARSRFLEWLLNYYGMDV

SEQ ID NO: 22 - protein, 112 aa, synthetic construct
DIVMTQSPLSLPVTPGEPASISCRSSQSLLHSNGYNYLDWYLQKPGQSPQFLIYLGSNRASGVPDRFSGSGSGTDFTLKISRVEAEDVGVYYCMQTLQTPPTFGQGTKVEIK

SEQ ID NO: 23 - protein, 11 aa, synthetic construct
QSLLHSNGYNY

SEQ ID NO: 24 - skipped

SEQ ID NO: 25 - protein, 9 aa, synthetic construct
MQTLQTPPT

SEQ ID NO: 26 - protein, 120 aa, synthetic construct
QVQLVQSGAEVKKPGASVKVSCKASGYNFAGYYMHWVRQAPGQGLEWMGWISPSSGGTNYAQKFQGRVTMTRDTSISTAYMELSRLRSDDTAVYYCASGGVVVVAAFDYWGQGTLVTVSS

SEQ ID NO: 27 - protein, 8 aa, synthetic construct
GYNFAGYY

SEQ ID NO: 28 - protein, 8 aa, synthetic construct
ISPSSGGT

SEQ ID NO: 29 - protein, 13 aa, synthetic construct
ASGGVVVVAAFDY

SEQ ID NO: 30 - protein, 112 aa, synthetic construct
QSVLTQPPSVSGAPGQRVTISCTGSSSNIGTGYDVHWYQQLPGTAPKLLIYNHSHRPSGVPDRFSGSKSGTSASLAITGLQAEDEADYYCQSYDSSLSGSWVFGGGTKLTVL

SEQ ID NO: 31 - protein, 9 aa, synthetic construct
SSNIGTGYD

SEQ ID NO: 32 - skipped

SEQ ID NO: 33 - protein, 12 aa, synthetic construct
QSYDSSLSGSWV

SEQ ID NO: 34 - protein, 118 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHSHWGFLDYWGQGTLVTVSS

SEQ ID NO: 35 - protein, 8 aa, synthetic construct
GYSFTSYW

SEQ ID NO: 36 - protein, 8 aa, synthetic construct
IYPGDSDT

SEQ ID NO: 37 - protein, 11 aa, synthetic construct
ARHSHWGFLDY

SEQ ID NO: 38 - protein, 112 aa, synthetic construct
DIVMTQSPLSLPVIPGEPASISCRSSQSLLQSNGYNYLDWYLQKPGQSPQLLIYLGSNRASGVPDRFSGSGSGTDFTLKISRVEAEDVGVYYCMQVQQTPYTFGQGTKLEIK

SEQ ID NO: 39 - protein, 11 aa, synthetic construct
QSLLQSNGYNY

SEQ ID NO: 40 - skipped

SEQ ID NO: 41 - protein, 9 aa, synthetic construct
MQVQQTPYT

SEQ ID NO: 42 - protein, 118 aa, synthetic construct
QVQLQESGPGLVKPSETLSLTCTVSGGSISSYYWTWIRQPAGKGLEWIGRIYTSGSTDYNPSLKSRVTMSVDTSKNQFSLKLSSVTAADTAVYYCARYNWNDDAFDIWGQGTMVTVSS

SEQ ID NO: 43 - protein, 8 aa, synthetic construct
GGSISSYY

SEQ ID NO: 44 - protein, 7 aa, synthetic construct
IYTSGST

SEQ ID NO: 45 - protein, 12 aa, synthetic construct
ARYNWNDDAFDI

SEQ ID NO: 46 - protein, 112 aa, synthetic construct
DIVMTQSPLSLPVTPGEPASISCRSSQSLLHRNGYNYLDWYLQKPGQSPQLLIYLGSNRASGVPDRFSGSGSGTDFTLKISRVEAEDVGIYYCMQALQIPHTFGQGTKLEIK

SEQ ID NO: 47 - protein, 11 aa, synthetic construct
QSLLHRNGYNY

SEQ ID NO: 48 - skipped

SEQ ID NO: 49 - protein, 9 aa, synthetic construct
MQALQIPHT

SEQ ID NO: 50 - protein, 123 aa, synthetic construct
QVQLQESGPGLVKPSQTLSLTCTVSGGSISSGGYCWSWIRQHPGKGLEWIGYIYYSGSTYYNPSLKSRVTISEDTSKNQFSLKLSSVTAADTAVYYCARGGRWLQLRDVFDIWGQGTMVTVSS

SEQ ID NO: 51 - protein, 10 aa, synthetic construct
GGSISSGGYC

SEQ ID NO: 52 - protein, 7 aa, synthetic construct
IYYSGST

SEQ ID NO: 53 - protein, 15 aa, synthetic construct
ARGGRWLQLRDVFDI

SEQ ID NO: 54 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAAYHCQQHSSSPLTFGGGTKVEIK

SEQ ID NO: 55 - protein, 7 aa, synthetic construct
QSVSSSY

SEQ ID NO: 56 - skipped

SEQ ID NO: 57 - protein, 9 aa, synthetic construct
QQHSSSPLT

SEQ ID NO: 58 - protein, 120 aa, synthetic construct
QVQLQESGPGLVKPSESLSLTCTVSGDSISSYYWSWIRQPAGKGLEWIGRFYTSGRSNYNPSLKSRVTMAVDTSKNQFSLKLSSVTAADTAVYYCARDGFGSYFGSFDYWGQGTLVTVSS

SEQ ID NO: 59 - protein, 8 aa, synthetic construct
GDSISSYY

SEQ ID NO: 60 - protein, 7 aa, synthetic construct
FYTSGRS

SEQ ID NO: 61 - protein, 14 aa, synthetic construct
ARDGFGSYFGSFDY

SEQ ID NO: 62 - protein, 106 aa, synthetic construct
EIVMTQSPATLSVSPGERATLSCRASQSIISNLAWYQQKPGQAPRLLIYDASTRAAGIPARFSGSGSGTEFTLTISTLQSEDFAVYYCQQYNKWPTFGGGTKVEIK

SEQ ID NO: 63 - protein, 6 aa, synthetic construct
QSIISN

SEQ ID NO: 64 - skipped

SEQ ID NO: 65 - protein, 8 aa, synthetic construct
QQYNKWPT

SEQ ID NO: 66 - protein, 124 aa, synthetic construct
EVQLVESGGGLVKPGGSLRLSCAASGFTFSSYSMNWVRQAPGKGLEWVSSISSSSSYIYYTDSVKGRFTISRDNAKNSLYLQMNSLSAEDTAVYYCARDRYDRSGYYYVPFDQWGQGTLVTVSS

SEQ ID NO: 67 - protein, 8 aa, synthetic construct
GFTFSSYS

SEQ ID NO: 68 - protein, 8 aa, synthetic construct
ISSSSSYI

SEQ ID NO: 69 - protein, 17 aa, synthetic construct
ARDRYDRSGYYYVPFDQ

SEQ ID NO: 70 - protein, 107 aa, synthetic construct
DIQMTQSPSTLSASVGDRVTITCRASQSITSWLAWYQQKPGKAPKLLIYKASSLESGVPSRFSGSGSGTEFTLTISSLQPDDFATYYCQQYNSFSWTFGQGTKVEIK

SEQ ID NO: 71 - protein, 6 aa, synthetic construct
QSITSW

SEQ ID NO: 72 - skipped

SEQ ID NO: 73 - protein, 9 aa, synthetic construct
QQYNSFSWT

SEQ ID NO: 74 - protein, 119 aa, synthetic construct
QVQLHESGPGLVKPSETLSLTCTVSGGSISSYYWSWIRQPAGKGVEWIGRIYPSGSTNYNPSLKSRVIMSVDTSKSQFSLKLSSVTAADTAVYYCARYSGICYGAFDIWGQGTMVTVSS

SEQ ID NO: 75 - protein, 8 aa, synthetic construct
GGSISSYY

SEQ ID NO: 76 - protein, 7 aa, synthetic construct
IYPSGST

SEQ ID NO: 77 - protein, 13 aa, synthetic construct
ARYSGICYGAFDI

SEQ ID NO: 78 - protein, 108 aa, synthetic construct
EIVLTQSPGTLSLSPGERATLSCRASQSVSSSYLAWYQQKPGQAPRLLIYGASSRATGIPDRFSGSGSGTEFTLTISRLEPEDFAVYYCQQHGSSPRTFGQGTKVEIK

SEQ ID NO: 79 - protein, 7 aa, synthetic construct
QSVSSSY

SEQ ID NO: 80 - skipped

SEQ ID NO: 81 - protein, 9 aa, synthetic construct
QQHGSSPRT

SEQ ID NO: 82 - protein, 118 aa, synthetic construct
QVQLQESGPGLVKPSETLSLTCTVSGDSISSYFWTWIRQPAGKGLEWIGRIYTSGITNYNPSLKSRVTMSVDTSKNQFSLKLNSVTAADTAVYYCARDTSSVGEFDYWGQGTLVTVSS

SEQ ID NO: 83 - protein, 8 aa, synthetic construct
GDSISSYF

SEQ ID NO: 84 - protein, 7 aa, synthetic construct
IYTSGIT

SEQ ID NO: 85 - protein, 12 aa, synthetic construct
ARDTSSVGEFDY

SEQ ID NO: 86 - protein, 110 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVFGGGTKLTVL

SEQ ID NO: 87 - protein, 9 aa, synthetic construct
SSDVGGYNY

SEQ ID NO: 88 - skipped

SEQ ID NO: 89 - protein, 10 aa, synthetic construct
SSYAGSNNLV

SEQ ID NO: 90 - protein, 117 aa, synthetic construct
QVQLVQSGAEVKKPGASVKVSCKASGYTFTSYYMHWVRQAPGQGLEWMGIINPSGGSTSYAQKFQGRVSMTSDTSTSTVSMELSSLRSEDTAIYYCARELGWGSDYWGQGTLVTVSS

SEQ ID NO: 91 - protein, 8 aa, synthetic construct
GYTFTSYY

SEQ ID NO: 92 - protein, 8 aa, synthetic construct
INPSGGST

SEQ ID NO: 93 - protein, 10 aa, synthetic construct
ARELGWGSDY

SEQ ID NO: 94 - protein, 107 aa, synthetic construct
DIQMTQSPSAMSASVGDRVTITCRASQDIRNYLAWFQQKPGKVPKRLIYIASNLQSGVPSRFSGSGSGTEFTLTISSLQPEDFATYYCLQHNSYPYTFGQGTKLEIK

SEQ ID NO: 95 - protein, 6 aa, synthetic construct
QDIRNY

SEQ ID NO: 96 - skipped

SEQ ID NO: 97 - protein, 9 aa, synthetic construct
LQHNSYPYT

SEQ ID NO: 98 - protein, 118 aa, synthetic construct
QVQLQESGLGLVKPSETLSLTCTVSGGSISSYYWSWIRQPPGKGLEWIGYIYSSGSTNDNPSLKSRVTISVDTSKNQFSLRLSSVTVADTAVYYCARFNWGNDAFDIWGQGTMVTVSS

SEQ ID NO: 99 - protein, 8 aa, synthetic construct
GGSISSYY

SEQ ID NO: 100 - protein, 7 aa, synthetic construct
IYSSGST

SEQ ID NO: 101 - protein, 12 aa, synthetic construct
ARFNWGNDAFDI

SEQ ID NO: 102 - protein, 112 aa, synthetic construct
DIVMTQSPLSLPVTPGEPASISCRSSQSLLHRNGYNYLDWYLQKPGQSPQLLIYLGSNRASGVTDRFSGSGSGTDFTLKISRVEAEDVGVYYCMQALQTPFTFGPGTKVDIK

SEQ ID NO: 103 - protein, 11 aa, synthetic construct
QSLLHRNGYNY

SEQ ID NO: 104 - skipped

SEQ ID NO: 105 - protein, 9 aa, synthetic construct
MQALQTPFT

SEQ ID NO: 106 - protein, 117 aa, synthetic construct
QVQLVQSGAEEKKPGASVKVSCKASGYTFTNYYMHWVRQAPGQGLEWMGIINPSGGNTSYAQKFQGRVTMTRDTSTSTVYMELSSLRSEDTAVYYCARELGWGSDYWGQGTLVTVSS

SEQ ID NO: 107 - protein, 8 aa, synthetic construct
GYTFTNYY

SEQ ID NO: 108 - protein, 8 aa, synthetic construct
INPSGGNT

SEQ ID NO: 109 - protein, 10 aa, synthetic construct
ARELGWGSDY

SEQ ID NO: 110 - protein, 107 aa, synthetic construct
DIQMTQSPSAMSASIGDRVTITCRASQDISNYLAWFQQKPGKVPKRLIYVASGLQSGVPSRFSGSGSGTEFTLTISSLQPEDFATYYCLQHNSYPFTFGQGTKLEIK

SEQ ID NO: 111 - protein, 6 aa, synthetic construct
QDISNY

SEQ ID NO: 112 - skipped

SEQ ID NO: 113 - protein, 9 aa, synthetic construct
LQHNSYPFT

SEQ ID NO: 114 - protein, 117 aa, synthetic construct
QVQLQESGPGLVKPSETLSLTCTVSSGSISPYYWSWIRQPPGKGLEWIGYIYYRGTTNYNPSLQSRVTMSVDTSKNQFSLKLSSVTAADTAVYYCARAALGAVFDYWGQGTLVTVSS

SEQ ID NO: 115 - protein, 8 aa, synthetic construct
SGSISPYY

SEQ ID NO: 116 - protein, 7 aa, synthetic construct
IYYRGTT

SEQ ID NO: 117 - protein, 11 aa, synthetic construct
ARAALGAVFDY

SEQ ID NO: 118 - protein, 110 aa, synthetic construct
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAVFFGGGTNLTVL

SEQ ID NO: 119 - protein, 8 aa, synthetic construct
SSNIGNNY

SEQ ID NO: 120 - skipped

SEQ ID NO: 121 - protein, 11 aa, synthetic construct
GTWDSSLSAVF

SEQ ID NO: 122 - protein, 121 aa, synthetic construct
QVQLVQSGAEVKKPGASVKVSCKASGYTFTSYYIHWVRQAPGQGLEWMGIINPSGGSISYAQKFQGRVTLTRDTSTSIVYMELSSLRSEDTAVYYCARGGTYYYYHGMDVWGQGTTVTVSS

SEQ ID NO: 123 - protein, 8 aa, synthetic construct
GYTFTSYY

SEQ ID NO: 124 - protein, 8 aa, synthetic construct
INPSGGSI

SEQ ID NO: 125 - protein, 14 aa, synthetic construct
ARGGTYYYYHGMDV

SEQ ID NO: 126 - protein, 110 aa, synthetic construct
QSALTQPASVSGSPGQSITISCTGTSSDVGSYNLVSWYQQHPGKAPKLMIYEGSKRPSGVSNRFSGSKSGNTASLTISGLQAEDEADYYCCSYAGSSTWVFGGGTKLTVL

SEQ ID NO: 127 - protein, 9 aa, synthetic construct
SSDVGSYNL

SEQ ID NO: 128 - skipped

SEQ ID NO: 129 - protein, 10 aa, synthetic construct
CSYAGSSTWV

SEQ ID NO: 130 - protein, 118 aa, synthetic construct
EVQLVQSGAEVKKPGESLKISCKGSGYSFISHWIGWVRQMPGKGLEWMGIIYPGDSDTRYSPSFQGQVTISADKSISTAYLQWSSLKASDTAIYYCARYYYYYGMDVWGQGTTVTVSS

SEQ ID NO: 131 - protein, 8 aa, synthetic construct
GYSFISHW

SEQ ID NO: 132 - protein, 8 aa, synthetic construct
IYPGDSDT

SEQ ID NO: 133 - protein, 11 aa, synthetic construct
ARYYYYYGMDV

SEQ ID NO: 134 - protein, 107 aa, synthetic construct
EIVMTQSPVTLSVSPGERATLSCRASQSVSSNLAWYQQKPGQAPRFLIYGASTRTTGIPARFSGSGSGTEFTLTISSLQSEDFAVYYCQQYNNWPYTFGQGTKLEIK

SEQ ID NO: 135 - protein, 6 aa, synthetic construct
QSVSSN

SEQ ID NO: 136 - skipped

SEQ ID NO: 137 - protein, 9 aa, synthetic construct
QQYNNWPYT

SEQ ID NO: 138 - protein, 118 aa, synthetic construct
QVQLQESGPGLVKPSQTLSLTCTVSGGSISSGGHYWNWIRQHPGKGLEWIGYIYYSGTTYYNPSLKSRVTISVDTSKNQFSLKLSSVTAADTAVYYCAREVGIAFDYWGQGTLVTVSS

SEQ ID NO: 139 - protein, 10 aa, synthetic construct
GGSISSGGHY

SEQ ID NO: 140 - protein, 7 aa, synthetic construct
IYYSGTT

SEQ ID NO: 141 - protein, 10 aa, synthetic construct
AREVGIAFDY

SEQ ID NO: 142 - protein, 107 aa, synthetic construct
DIQLTQSPSFLSASVGDRVTITCRASQGVSSYLAWYQQKPGKAPKLLIFAASALQSGVPSRFSGSGSGTEFTLTISSLQPEDFATYHCQQLNSYPPTFGQGTKVEIK

SEQ ID NO: 143 - protein, 6 aa, synthetic construct
QGVSSY

SEQ ID NO: 144 - skipped

SEQ ID NO: 145 - protein, 9 aa, synthetic construct
QQLNSYPPT

SEQ ID NO: 146 - protein, 118 aa, synthetic construct
EVQLVQSGAEVKKPGESLKISCKGSGYNFPNYWIAWVRQMPGKGLEWMGIIYPGDSDTRYSPSFQGQVTISADKSISTAYLQWSSLKASDTAMYYCARHSNWGHFDYWGQGTLVTVSS

SEQ ID NO: 147 - protein, 8 aa, synthetic construct
GYNFPNYW

SEQ ID NO: 148 - protein, 8 aa, synthetic construct
IYPGDSDT

SEQ ID NO: 149 - protein, 11 aa, synthetic construct
ARHSNWGHFDY

SEQ ID NO: 150 - protein, 112 aa, synthetic construct
DIVMTQSPLSLPVTPGEPASISCRSSQSLLHSNGYNYLDWYLQKPGQSPQVLIYMGSNRASGVPDRFSGSGSGTDFTLKISRVEAEDVGVYYCMQAQQTPYTFGQGTKLEIK

SEQ ID NO: 151 - protein, 11 aa, synthetic construct
QSLLHSNGYNY

SEQ ID NO: 152 - skipped

SEQ ID NO: 153 - protein, 9 aa, synthetic construct
MQAQQTPYT

SEQ ID NO: 154 - protein, 120 aa, synthetic construct
EVQLVESGGGLVQPGGSLRLSCAASGFTFSSYWMSWARQAPGKGLEWVANIKQDGSEKYYVDSVKGRFTISRDNAKNSLYLQMNSLRVEDTAVYYCASSGIVGASTLDYWGQGILLIVSA

SEQ ID NO: 155 - protein, 8 aa, synthetic construct
GFTFSSYW

SEQ ID NO: 156 - protein, 8 aa, synthetic construct
IKQDGSEK

SEQ ID NO: 157 - protein, 13 aa, synthetic construct
ASSGIVGASTLDY

SEQ ID NO: 158 - protein, 108 aa, synthetic construct
EIVMTQSPATMSVSPGERATLSCRASQSVSSNLAWFQQKPGQAPRLLIYGASTRATGIPARFSGSGSGTEFTLTISSLQSEDFAVYYCQQYNNWPPYTFGQGTKLEIK

SEQ ID NO: 159 - protein, 6 aa, synthetic construct
QSVSSN

SEQ ID NO: 160 - skipped

SEQ ID NO: 161 - protein, 10 aa, synthetic construct
QQYNNWPPYT

SEQ ID NO: 162 - protein, 119 aa, synthetic construct
EVQLVESGGGLVQPGGSLRLSCAASGFTFSSYRLNWVRQAPGKGLEWVSYISSSTGSLDYADSVKGRFTISRDNAKNSLYLQMNSLRDEDTAVYYCARRSPYTGSLDNWGQGTLVTVSS

SEQ ID NO: 163 - protein, 8 aa, synthetic construct
GFTFSSYR

SEQ ID NO: 164 - protein, 8 aa, synthetic construct
ISSSTGSL

SEQ ID NO: 165 - protein, 12 aa, synthetic construct
ARRSPYTGSLDN

SEQ ID NO: 166 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKINRVEAEDVGVYYCMQALQTPYTFGQGTKLEIK

SEQ ID NO: 167 - protein, 11 aa, synthetic construct
QSLLHSNGYNY

SEQ ID NO: 168 - skipped

SEQ ID NO: 169 - protein, 9 aa, synthetic construct
MQALQTPYT

SEQ ID NO: 170 - protein, 117 aa, synthetic construct
QVQLVQSGAEVKKPGSSVKVSCKASGGTFSSYGISWVRQAPGQGLEWMGGIIPFFGSANYAQKFQGRVTITADKSTSTAYMELSSLRSEDTAVYYCARDGGAILDYWGQGTLVTVSS

SEQ ID NO: 171 - protein, 8 aa, synthetic construct
GGTFSSYG

SEQ ID NO: 172 - protein, 8 aa, synthetic construct
IIPFFGSA

SEQ ID NO: 173 - protein, 10 aa, synthetic construct
ARDGGAILDY

SEQ ID NO: 174 - protein, 106 aa, synthetic construct
SYELTQPSSVSVSPGQTARITCSGDVLAKKYARWFQQKPGQAPLLVIYKDSERPSGIPERFSGSSSGTTVTLTISGAQVEDEADYYCYSAADNNWVFGGGTKLTVL

SEQ ID NO: 175 - protein, 6 aa, synthetic construct
VLAKKY

SEQ ID NO: 176 - skipped

SEQ ID NO: 177 - protein, 9 aa, synthetic construct
YSAADNNWV